(19) United States
(12) Patent Application Publication
(10) Pub. No.: US 2012/0203300 A1
(43) Pub. Date: Aug. 9, 2012

(54) Method and Apparatus for Ablation of Benign, Pre-Cancerous and Early Cancerous Lesions That Originate Within the Epithelium and are Limited to the Mucosal Layer of the Gastrointestinal Tract

(75) Inventors: David S. Utley, Redwood City, CA (US); Michael P. Wallace, Pleasanton, CA (US)

(73) Assignee: BARRX Medical, Inc., Sunnyvale, CA (US)

(21) Appl. No.: 13/438,652

(22) Filed: Apr. 3, 2012

Related U.S. Application Data
(62) Division of application No. 12/168,042, filed on Jul. 3, 2008.
(60) Provisional application No. 60/958,562, filed on Jul. 6, 2007.
(60) Provisional application No. 60/958,566, filed on Jul. 6, 2007.

(51) Int. Cl. A61B 18/12

(52) U.S. Cl. 607/40

(57) ABSTRACT

Devices and methods are provided for ablating areas of the gastrointestinal tract affected with certain benign, pre-cancerous, or early cancerous lesions that originate within the epithelium and are limited to the mucosal layer of the gastrointestinal tract wall. Examples of such lesions include benign conditions such as cervical inlet patch (ectopic gastric mucosa in the upper esophagus), as well as pre-cancerous and cancerous conditions such as intestinal metaplasia/intra-epithelial neoplasia/early cancer of the stomach, squamous intra-epithelial neoplasia and early cancer of the esophagus, oral and pharyngeal leukoplakia, flat colonic polyps, anal intra-epithelial neoplasia (AIN), and early cancers of the anal canal. Ablation, as provided in an embodiment of the invention, commences at the epithelial layer of the gastrointestinal wall and penetrates deeper into the gastrointestinal wall in a controlled manner to achieve a successful patient outcome.

### CROSS REFERENCE TO RELATED APPLICATIONS

This application claims the benefit of U.S. Provisional Patent Application No. 60/958,562 “Non-Barrett's Mucosal Ablation Disease Targets,” by Utley and Wallace, as filed on Jul. 6, 2007, and of U.S. Provisional Application No. 60/958,566, entitled “Non-Barrett's Mucosal Ablation Disease Targets” by Utley et al., as filed on Jul. 6, 2007.

This application also incorporates herein by reference commonly assigned U.S. patent application Ser. No. 10/370,645 entitled “Method of Treating Abnormal Tissue in the Human Esophagus,” filed on Feb. 19, 2003, and published as US 2003/0158550 on Aug. 21, 2003, and U.S. patent application Ser. No. 11/286,444 entitled “Precision Ablating Method,” filed on Nov. 23, 2005, and published as US 2007/0118106 on May 24, 2007. Further, each of the following commonly assigned U.S. patent applications are incorporated herein by reference in its entirety: patent application Ser. No. 10/291,862 titled “Systems and Methods for Treating Obesity and Other Gastrointestinal Conditions,” patent application Ser. No. 10/370,645 titled “Method of Treating Abnormal Tissue In The Human Esophagus,” patent application Ser. No. 11/286,257 titled “Precision Ablating Device,” patent application Ser. No. 11/275,244 titled “Auto-Aligning Ablating Device and Method of Use,” patent application Ser. No. 11/286,444 titled “Precision Ablating Device,” patent application Ser. No. 11/420,712 titled “System for Tissue Ablation,” patent application Ser. No. 11/420,714 titled “Method for Cryogenic Tissue Ablation,” patent application Ser. No. 11/420,719 titled “Method for Vacuum-Assisted Tissue Ablation,” patent application Ser. No. 11/420,722 titled “Method for Tissue Ablation,” patent application Ser. No. 11/469,816 titled “Surgical Instruments and Techniques for Treating Gastro-esophageal Reflux Disease.” This application further incorporates in entirety U.S. patent application Ser. No. 10/291,862 of Utley, filed on Nov. 8, 2002 entitled “Systems and Methods for Treating Obesity and Other Gastrointestinal Conditions,” and published on May 13, 2004 as US 2004/0089313, and U.S. Pat. No. 7,326,207 of Edwards, entitled “Surgical Weight Control Device,” which issued on Feb. 5, 2008. This application further incorporates in entirety U.S. patent application Ser. No. 12/114,628 of Kelly et al. entitled “Method and Apparatus for Gastrointestinal Tract Ablation for Treatment of Obesity,” as filed on filed May 2, 2008. This application further incorporates in entirety U.S. patent application Ser. No. 12/143,404, of Wallace et al., entitled “Electrical Means to Normalize Ablational Energy Transmission to a Luminal Tissue Surface of Varying Size,” as filed on Jun. 20, 2008.

### INCORPORATION BY REFERENCE

All publications, patents and patent applications mentioned in this specification are herein incorporated by reference to the same extent as if each individual publication, patent or patent application was specifically and individually indicated to be incorporated by reference.

### FIELD OF THE INVENTION

The present invention relates to therapeutic devices and methods for treatment of the gastrointestinal tract affected with certain benign, pre-cancerous and early cancerous lesions that originate within the epithelium and are limited to the mucosal layer of the gastrointestinal tract wall.

### BACKGROUND OF THE INVENTION

There is yet to be an ideal, non-surgical, therapeutic intervention for certain benign, precancerous and early cancerous lesions that originate within the epithelium and are limited to the mucosal layer of the gastrointestinal tract wall. Examples of such lesions are illustrated schematically in FIG. 1 and include benign (non-cancerous) conditions such as cervical inlet patch (ectopic gastric mucosa in the upper esophagus), as well as pre-cancerous and cancerous conditions such as intestinal metaplasia/intra-epithelial neoplasia/early cancer of the stomach, squamous intra-epithelial neoplasia and early cancer of the esophagus, oral and pharyngeal leukoplakia, flat colonic polyps, anal intra-epithelial neoplasia (AIN) and early cancers of the anal canal. These benign, precancerous and cancerous lesions of the gastrointestinal tract that originate within the epithelium are potentially amenable to curative endoscopic therapy, as described herein, thus avoiding major surgery. Certain non-surgical therapies have been attempted for treating these lesions, however, they have been limited in safety, technical feasibility, and effectiveness.

A common denominator for these lesions is that they originate within the epithelium, the most superficial layer of the gastrointestinal tract wall. For the benign and pre-cancerous lesions, the only layer affected by the lesion is the epithelium, making these lesions highly amenable to an optimized endoscopic therapy, as disclosed herein. For certain other types of lesions, such as early stage cancerous lesions that are limited to the mucosal layer (epithelium, lamina propria, and muscularis mucosae), curative therapy is also possible using an optimized therapy, as disclosed herein.

While these lesions have the described common denominator of originating within the epithelial layer, an important differentiating feature is that they occur in different regions of the gastrointestinal tract which have different anatomic and geometric configurations, thus mandating different devices and methods to achieve effective treatment.

A further differentiating feature for these lesions is that they each have diverse etiology, lesion characteristics (depth, for example), and propensity to cause patient morbidity and mortality. Cervical inlet patch of the esophagus is an embryological remnant of gastric tissue that resides high in the esophagus. Often, this lesion produces stomach acid, thereby causing discomfort in the esophagus. Intestinal metaplasia, intra-epithelial neoplasia, and early cancer of the stomach are a spectrum of progressive tissue changes towards invasive gastric cancer, a world-wide epidemic. These changes may be caused by diet, smoking, and infection. Squamous intra-epithelial neoplasia and early cancer of the esophagus is related to diet, environmental fungus, smoke exposure, and alcohol use, and is also a world-wide epidemic. Oral and pharyngeal leukoplakia is an epithelial pre-cancerous change that leads to head and neck squamous cell cancer, and is due to smoking and alcohol use.

Flat polyps of the colon and rectum are precursors to more advanced polypoid lesions and invasive cancer. FIGS. 2A, 2B and 2C are schematic side views of colorectal polyps 52 or adenomas referred to collectively herein as flat type polyps. The exemplary polyps are stalked (FIG. 2A), pedunculated (FIG. 2B) and sessile (FIG. 2C). Anal intra-epithelial neoplasia and early cancer of the anal canal occurs as a result of human papilloma infection predominantly in men who have sex with men.

There remains a need for improved non-surgical, therapeutic intervention for certain benign, precancerous and early cancerous lesions that originate within the epithelium and are limited to the mucosal layer of the gastrointestinal tract wall.

### SUMMARY OF THE INVENTION

Provided herein by the invention are methods for ablation therapy directed to benign, pre-cancerous and early cancerous lesions of the gastrointestinal tract that originate within the epithelium and are contained within the mucosal layer. Such lesions may include, by way of example, a cervical inlet patch within a portion of the proximal esophagus, abnormal gastric tissue (such as intestinal metaplasia, intraepithelial neoplasia, and early cancer of the stomach), abnormal esophageal tissue (such as squamous intraepithelial neoplasia and early cancer), leukoplakia within the oral or pharyngeal cavity, polyps in the colon or rectum, anal lesions (such as anal intraepithelial neoplasis and early anal cancer). These lesions, in spite of differences in particulars of origin, developmental stage, and morphology, for the purpose of this summary, will be collectively referred to as lesions within the mucosal layer of the gastrointestinal tract.

Embodiments of the method of ablation therapy directed to a target area of a lesion within the mucosal layer of the gastrointestinal traction include manipulating a portion of the gastrointestinal tract near the lesion in order to expose the target area, deploying or advancing an ablation device into contact with the target area, delivering ablative energy to a tissue surface in the target area; and controlling the delivery of ablative energy to the tissue surface and into tissue layers of the target area.

The method may further include, in addition to or in conjunction with the manipulating step, any of identifying the lesion, identifying a target area within the lesion, or manipulating the lesion site or target area in order to expose the target area during the steps of delivering of ablative energy and controlling the delivery of ablative energy.

The method may further include removing debris from the target area after the delivering and controlling steps, and it may further include removing debris from the target area after performing the controlling step more than once.

The step of controlling the delivery of energy may include controlling the energy density such that it is in the range of about 10-to about 15 J/cm².

The step of delivering energy may include delivering ablative energy without an electrode structure penetrating tissue in the target area.

The step of controlling the delivery of energy may include delivering sufficient ablative energy to achieve ablation in one fraction of the lesion's tissue target surface and delivering insufficient ablative energy to achieve ablation to another fraction of the lesion's target tissue surface. The step of controlling the delivery of energy may also include controlling the delivery of ablative energy to the lesion's target tissue surface to provide sufficient treatment to achieve ablation within tissue layers near the surface of the target area and yet provide insufficient energy to deeper tissue layers beneath the target area of the lesion.

In another aspect, controlling the delivery of ablative energy across the surface and into tissue layers in the lesion's target area is such that some fraction of the tissue volume is ablated and another fraction of the tissue volume is not ablated. Thus, with more specific regard to the tissue layers of the lesion, controlling the delivery of energy into target tissue layers may variously consist of ablating a fraction of tissue in the epithelial layer of the cervical inlet patch, ablating a fraction of tissue in the epithelial layer and the lamina propria of the cervical inlet patch, ablating a fraction of cervical inlet patch tissue in the epithelial layer, the lamina propria, and the muscularis mucosae, or ablating a fraction of cervical inlet patch tissue in the epithelial layer, the lamina propria, the muscularis mucosae, and the submucosa.

In some embodiments of the method, the delivering energy step may further include delivering energy in an ablation pattern that conforms to the specific size and conformational features of the lesion, such size and conformation being particular to each lesion addressed by the method, such as a cervical inlet patch within a portion of the proximal esophagus, an abnormal gastric tissue (such as intestinal metaplasia, intra-epithelial neoplasia, and early cancer of the stomach), an abnormal esophageal tissue (such as squamous intra-epithelial neoplasia and early cancer), a site of leukoplakia within the oral or pharyngeal cavity, polyps in the colon or rectum, and anal lesions (such as anal intraepithelial neoplasis and early anal cancer). Such particulars of lesion size and conformation may include, for example, lesions being flat, as oral leukoplakia are, or stalked or pedunculated as some colorectal polyps may be, or particulars such as the size and available capacity for instrument maneuverability as in the stomach, or the relative accessibility of the anus or oral cavity.

Some embodiments of the method may further include evaluating the target area of the lesion at a point in time after the delivering energy step, in order to determine the status of the area. The evaluating step may occur in close time proximity after the delivery of energy, to evaluate the immediate post-treatment status of the site. In various embodiments, the evaluating step occurs at least one day after the delivery of energy.

In some embodiments of the method, the controlling step may further include adjusting the energy delivery to provide any of a specific power, a power density, an energy level, an energy density, a circuit impedance, target tissue temperature, a number of applications of energy, or a pressure of application against the tissue.

In some embodiments of the method, the deploying or advancing step may further include moving the ablation structure into therapeutic contact with the target area prior to the delivering energy step. In various embodiments, the moving step may include expanding an expandable member to enhance the therapeutic contact with the target tissue, or operating a deflection mechanism to enhance the therapeutic contact with the target tissue.

### BRIEF DESCRIPTION OF THE DRAWINGS

FIG. 1 provides a view of the gastrointestinal tract and the location of sites of abnormal tissue that may be targeted for ablation, as provided by systems and methods of ablation as described herein.

FIGS. 2A, 2B and 2C are schematic side views of colorectal polyps or adenomas referred to collectively as flat type polyps. The exemplary polyps are stalked, pedunculated and sessile, respectively.

FIG. 3 is a flow diagram depicting an overview of the method, wherein an appropriate site for ablational treatment of a gastrointestinal tract having one or more abnormal lesions is determined, the level of ablational therapy is determined, and at least preliminary information is gained regarding localization, and clinical judgment is exercised as to which embodiment of the invention is preferable.

FIG. 4 is a flow diagram depicting the method after the site of ablation of a portion of the gastrointestinal tract has been localized and a choice has been made regarding the preferred ablational device. The method includes an evaluation of the site, including particulars of location, stage, determination of the number of sites, and the dimensions. The method continues with insertion of the instrument and its movement to the locale of the ablational target tissue, the more refined movement of the ablational structure that create a therapeutically effective contact, the emission of ablational radiation and then post-treatment evaluation.

FIG. 5 is a view of an embodiment of an ablative device with a fully circumferential operating radius.

FIG. 6 is a view of an embodiment of an ablative device with a fully circumferential operating radius, with a balloon member in an expanded configuration.

FIGS. 7A-7C show the electrode patterns of the device of FIG. 5.

FIGS. 8A-8D show electrode patterns that may be used with embodiments of the ablative device with a fully circumferential operating radius, or with any device embodiments described herein.

FIG. 9 is a view of the ablation device of the invention with a partially circumferential operating radius.

FIG. 10 is an end view of the ablation device embodiment of FIG. 9.

FIG. 11 is an end view of the device of FIG. 9 in an expanded configuration.

FIGS. 12, 13, and 14 are end views of the device of FIG. 9 in alternative expanded configurations.

FIG. 15 is a view of the ablation device of the invention in an unexpanded configuration.

FIG. 16 is a view of the ablation device of the invention in an expanded configuration.

FIGS. 17 and 18 are end views of the device in an expanded configuration.

FIG. 19A is a view of the ablation device of the invention showing a deflection member feature.

FIG. 19B is a view of the ablation device of the invention showing an alternative deflection member wherein the device is in an expanded configuration.

FIG. 20 is a view of device shown in FIG. 19B wherein the deflection member is in an unexpanded configuration.

FIG. 21 is an end view of the device in an unexpanded configuration.

FIG. 22 is an end view of the device shown in FIG. 21 in an expanded configuration.

FIG. 23 is a view of the ablation device of the invention showing a pivoting ablation structure feature.

FIG. 24 is an illustration of the ablation device of the invention combined with an endoscope system.

FIG. 25 is a schematic of view of a section through the wall of a representative organ of the gastrointestinal tract.

FIG. 26 is a view of the ablation device of the invention including an elongated sheath feature.

FIG. 27 is a view of the device wherein an elongated sheath feature is optically transmissive.

FIG. 28 is an enlarged view of the optically transmissive feature of the device.

FIG. 29 is a cross sectional view of the optically transmissive sheath feature of the device shown in FIGS. 27 and 28.

FIG. 30 is a view of the device including an alternative optically transmissive sheath feature and an inflation member feature in an expanded configuration.

FIG. 31 is an illustration of the ablation device of FIG. 30 positioned within an esophagus.

FIG. 32 is a view of the ablation device of the invention including a slit sheath feature.

FIG. 33A is an end view of a slit sheath feature of the device wherein the sheath is in an unexpanded configuration.

FIG. 33B is an end view of a slit sheath feature of the device and an endoscope wherein the sheath is in an expanded configuration.

FIG. 34A is a cross sectional view of the device positioned within an endoscope internal working channel wherein an inflatable member feature is in an unexpanded position.

FIG. 34B is a view of the device shown in FIG. 34A wherein the inflatable member feature is in an expanded position.

FIG. 35A is a cross sectional view of the device positioned within an endoscope internal working channel wherein an expandable member feature is in an unexpanded position.

FIG. 35B is a view of the device shown in FIG. 35A wherein the expandable member feature is in an expanded position.

FIG. 36A is a cross sectional view of the device positioned within an endoscope internal working channel wherein an alternative expandable member feature is in an unexpanded position.

FIG. 36B is a view of the device shown in FIG. 36A wherein the expandable member feature is in an expanded position.

FIG. 37 is a view of the ablation device of the invention including an alternative deflection member.

FIG. 38 is an illustration of the ablation device of the invention including an alternative deflection member positioned within the lumen of an organ of the gastrointestinal tract in a non-deflected position.

FIG. 39 is an illustration of the device shown in FIG. 38 wherein the deflection member is in a deflected position.

FIG. 40 is a cross sectional view of the ablation device of the invention showing an internal coupling mechanism feature.

FIG. 41 is a cross sectional view of the ablation device of the invention showing an alternative internal coupling mechanism and a rolled sheath feature.

FIG. 42 is an illustration showing a cross sectional view of the ablation device of the invention positioned within the lumen of an organ of the gastrointestinal tract.

FIG. 43 is an illustration of the ablation device of the invention positioned within an esophagus showing a rotational feature.

FIG. 44 is an illustration of the ablation device of the invention positioned within an esophagus showing a rotational feature combined with an inflation member in an expanded configuration.

FIGS. 45A-45C are views of the ablation device of the invention showing alternative rotational features.

FIG. 46A is a view of an endoscope.

FIG. 46B is a view of the ablation device of the invention including a catheter feature.

FIG. 46C is a view of a sheath feature of the device.

FIG. 47 is a view of the ablation device of the invention including the features shown in FIGS. 46A-46C in an assembly.

FIGS. 48A-48D show an electrode array with a striped pattern for a fractional ablation and the ablation patterns on tissue that can be made from such a pattern.

FIGS. 49A and 49B show an electrode array with a concentric-circle pattern for a fractional ablation and the ablation patterns on tissue that can be made from such a pattern.

FIGS. 50A and 50B show an electrode array with a checkerboard pattern for a fractional ablation and the ablation patterns on tissue that can be made from such a pattern.

FIGS. 51A and 51B show an electrode array with a checkerboard pattern operating in a non-fractional manner and the ablation pattern on tissue that is made from such an operating pattern.

FIGS. 52A and 52B show an electrode array with a checkerboard pattern operating in a fractional manner and the ablation pattern on tissue that is made from such an operating pattern.

FIGS. 53A and 53B show an electrode array with a striped pattern of alternating positive and negative electrodes operating in a non-fractional manner and the ablation patterns on tissue that can be made from such an operating pattern.

FIGS. 54A and 54B show an electrode array with a striped pattern of alternating positive and negative electrodes operating in a fractional manner and the ablation patterns on tissue that can be made from such an operating pattern.

FIG. 55 shows a schematic rendering of a three-dimensional view of a target region of a radial portion of a gastrointestinal wall after it has been ablationally treated.

FIGS. 56A and 56B provide views of an ablational device (similar to the devices of FIGS. 38 and 39) but including an ablational surface on a hinge structure or deflecting mechanism similar to that depicted in FIG. 43, the hinge allowing a free pivoting movement of the ablational surface between its longitudinal axis and the longitudinal axis of an endoscope. FIG. 56A shows the device with the ablational surface oriented in parallel with the endoscope. FIG. 56B shows the device with the longitudinal axis of the ablational surface oriented at about a right angle with respect to the longitudinal axis of the endoscope.

FIG. 57A-57D provide perspective views of an ablation device with a 360 degree circumferential ablation surface on an overlapping electrode support furled around an expandable balloon, the operative element including a balloon and an electrode support in an expanded state. FIG. 57A shows the support pulled away from the balloon to clarify that a portion of the support and an edge is adherent to the balloon, and another portion and its edge is not connected to the balloon.

FIG. 57B shows the operative element of the device with the non-adherent portion of the support furled around the balloon in a deployable configuration, the non-adherent portion and its edge overlapping around the adherent portion.

FIG. 57C shows the device of FIGS. 57A and 57B with an optional feature of the operative element, one or more elastic bands wrapped around the electrode support.

FIG. 57D shows the device of FIG. 57C in a collapsed state, with balloon portion being uninflated (or deflated), this being the state of the device when it is being deployed into a lumen and being positioned at a target site, as well as the state of the device after delivering ablation energy and about to be removed from the lumen.

FIGS. 58A-58B depict an embodiment of an ablation device that is adapted to present an ablational surface into a concave or inwardly tapered target site such as the pylorus. The device includes an ablational surface circumferentially arranged on the distal portion of an expandable member, the expandable member mounted around the distal end of an endoscope. FIG. 58A shows the device in a deployed configuration.

FIG. 58B shows the device with the expandable member in an unexpanded or collapsed state, as would be appropriate for deployment of the device to a target tapered surface, or as would be appropriate for removal from the ablational site.

FIG. 59 is a perspective view of a hand held ablation therapy device.

FIG. 60 is a perspective view of a finger-mountable ablation therapy device.

### DETAILED DESCRIPTION

Aspects of the present invention provide various embodiments of a therapeutic device and method to treat the disclosed benign, pre-cancerous and early cancerous lesions that originate within the epithelium and are limited to the mucosal layer of the gastrointestinal tract wall. Successful treatment of these lesions implies a treatment that does not cause excessive patient morbidity due to over-treatment, excessively deep penetration of the treatment effect, perforation, bleeding, or other such complication. A successful treatment, from an efficacy standpoint, is defined as complete removal of all abnormal tissue, a change in the abnormal tissue such that it no longer produces symptoms, or change in the abnormal tissue such that it no longer has the propensity to develop invasive cancer or that the risk of developing invasive cancer is more remote or time-delayed.

Current techniques, excluding the present invention disclosed herein, for treating these disclosed lesions include coagulation, mucosal resection, and cryotherapy. These techniques are limited in the amount of tissue surface area that can be safely and effectively treated during one or more treatment sessions, due to specific limitations of the device, technique, and tissue effects, so wide-spread lesions are not amenable to effective treatment with these approaches. Further, coagulation and cryotherapy are limited in their ability to control the depth of ablation, resulting in under-treatment and over-treatment of certain areas within the lesion. This non-uniform treatment can result in persistence of the lesion (under-treatment) or patient complications (over-treatment). Mucosal resection is a deep resection technique that removes the entire mucosa and submucosa, a depth of penetration that is excessive and unnecessary for the successful treatment of the disclosed lesions. Wide-spread endoscopic resection can result in significant complications and is not feasible in most eases.

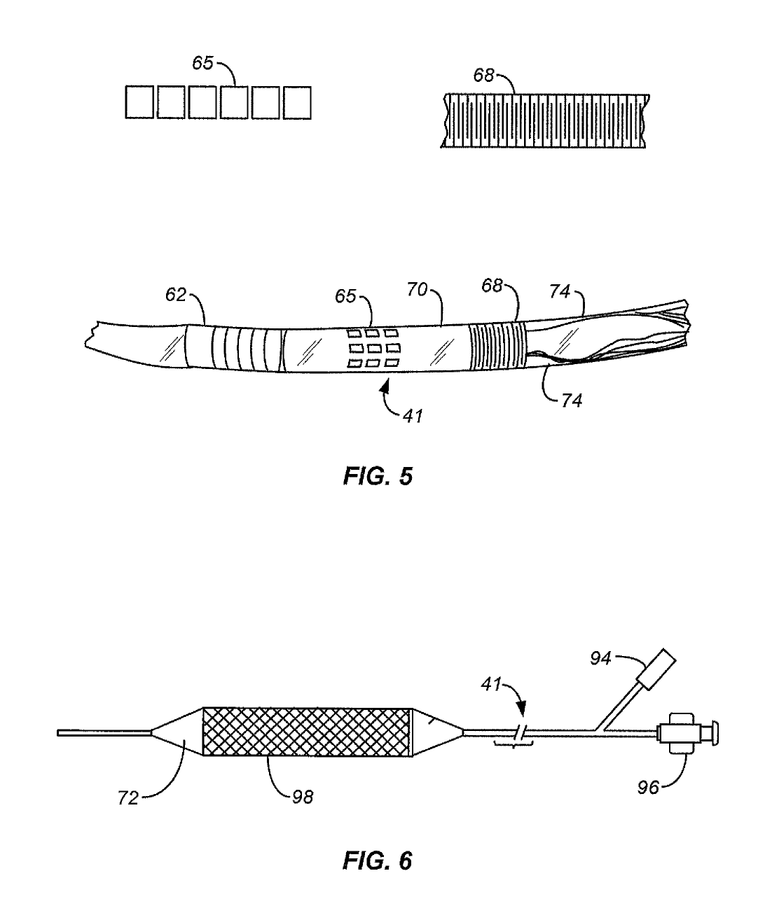

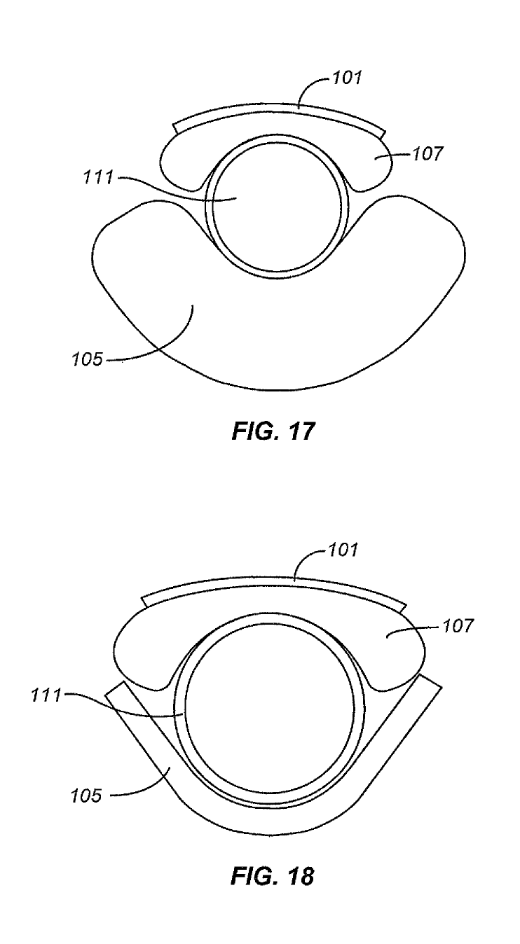

To this end, in some the device embodiments disclosed herein there is a catheter that is either balloon-based or not balloon-based, and, is either mounted on the end of an endoscope, passes through a working channel or accessory channel of an endoscope, passes along side an endoscope, or is hand-held using direct visualization of the target lesion. Alternatively, the device may be handheld or worn on one or more fingers. The device has an energy delivery element, such as an electrical array, on at least one surface to deliver ablation energy from a source to the targeted tissue in a manner so that the depth of ablation is controlled via parameters such as energy density, electrode pattern, power density, number of applications, and pressure exerted on the tissue. This configuration allows both successful treatment of focal lesions as well as successful treatment of more widespread, diffuse lesions. The catheter is supplied with ablation energy by an energy generator, connected to the catheter with a cable. Various alternative ablation devices are illustrated and described with regard to FIGS. 5 to 60.

Embodiments of the inventive method includes using the devices described, in conjunction with an endoscope for visualization of the lesion in seine cases (or for some lesions, using direct visualization without an endoscope), positioning the device in one or more locations at the target lesion, deploying the device so as to make therapeutic contact with the lesion, and delivering ablative energy one or more times. Treatment parameters may be such that a uniform level of ablation is achieved in all or part of the lesion. For example, the entire epithelium can be removed, without injury to deeper layers of the structure. Another example is to apply energy in a uniform manner to incur a deeper injury, including the entire thickness of the mucosa (epithelium, lamina propria, muscularis mucosae). Yet another example would be to apply the treatment to include a portion of the submucosa. The desired depth of ablation and pattern of ablation is predicated upon the specific lesion being treated.

One factor for successful treatment of these lesions is adequate contact of the treatment element with the lesion and the epithelial surface. In some circumstances, this therapeutic contact can be achieved using a relatively planar structure mounted on the end of an endoscope (for lesions in the stomach or esophagus, for example). In other circumstances involving tubular structures, this may be achieved with a balloon-mounted treatment element (as in the proximal esophagus or colon, for example). In other circumstances, a more complex anatomic and geometric structure must be treated, requiring the treatment element to be mounted on a conformable structure, such as a malleable substrate, or alternatively a sponge (as in lesions located in the oral cavity, pharyngeal space, distal rectum and anal canal.)

A number of embodiments of ablation devices are provided herein, which may be described as having an ablational surface that spans either a 360-degree circumference, or some fractional portion of a full circumference around the device. For example, some devices have an ablational surface that spans about 180 degrees, and others have an ablational surface that spans about 90 degrees. Ablation devices may be mounted on an instrument such as a catheter, endoscope or colonscope. Some ablation device embodiments are hand held or worn on one or more fingers or a glove worn by a user. The ablation devices may be used to accomplish the method treatment described herein.

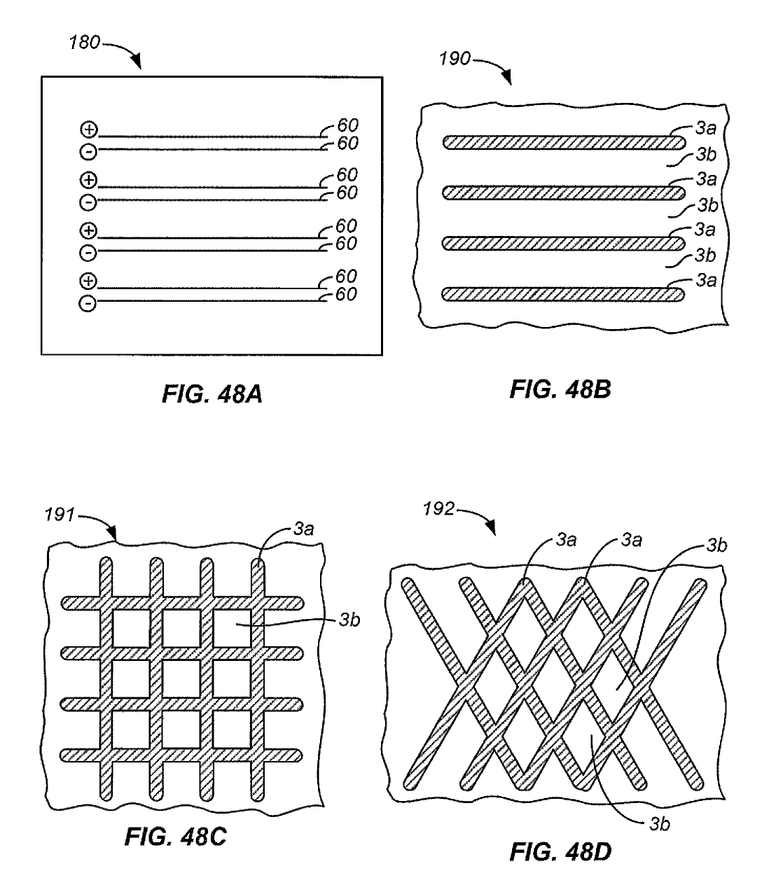

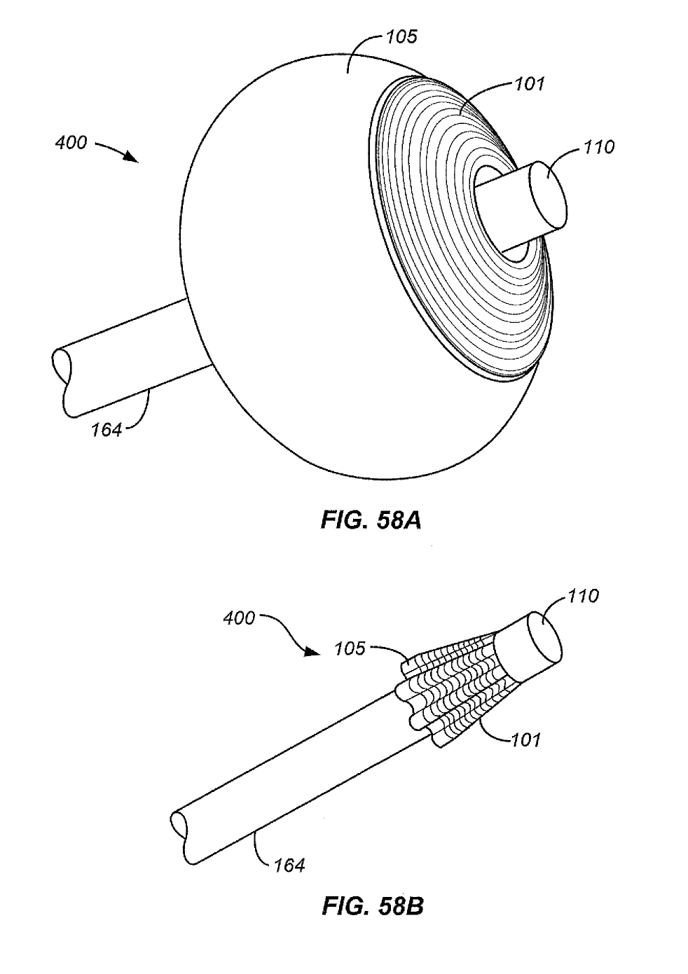

The various alternative device embodiments may be categorized based on the size of the ablation device and the configuration of the ablational surface. Some device embodiments have an ablation surface that spans a complete 360 degree circumference that is expandable through the use of an expandable member included in the device internal to the ablational surface. Several such representative embodiments are shown and further described below (FIGS. 6, 57, and 58) and described further below. Embodiments of the fully circumferential ablational surface are typically cylindrical in form, but embodiments can include circumferential ablational surfaces arranged on surfaces that depart from strict cylindrical, and become more ovalular or spherical, as shown in FIGS. 58A and 58B, with one or both of the (proximal or distal) ends being tapered. By way of further description of the ablational surface, it includes ablational delivery elements such as non-penetrating radiofrequency electrodes, but other types of ablational energy elements are includes as embodiments as well, and as described further below. Exemplary arrangements of radiofrequency electrodes are shown in FIGS. 5, and 7-9. Arrangements of energy delivery elements that create a fractional or partial ablation within a target area, as well as the ablation patterns they deliver to target tissue, are described further below, and depicted in FIGS. 48-55. Another feature shared by energy delivery element patterns provided herein is that although the ablation pattern is on a surface that may be pressed into therapeutic contact by an expandable member, the immediate surface upon which the energy delivery elements are arranged is substantially non-distensible, thus the density of elements across the surface remains constant.

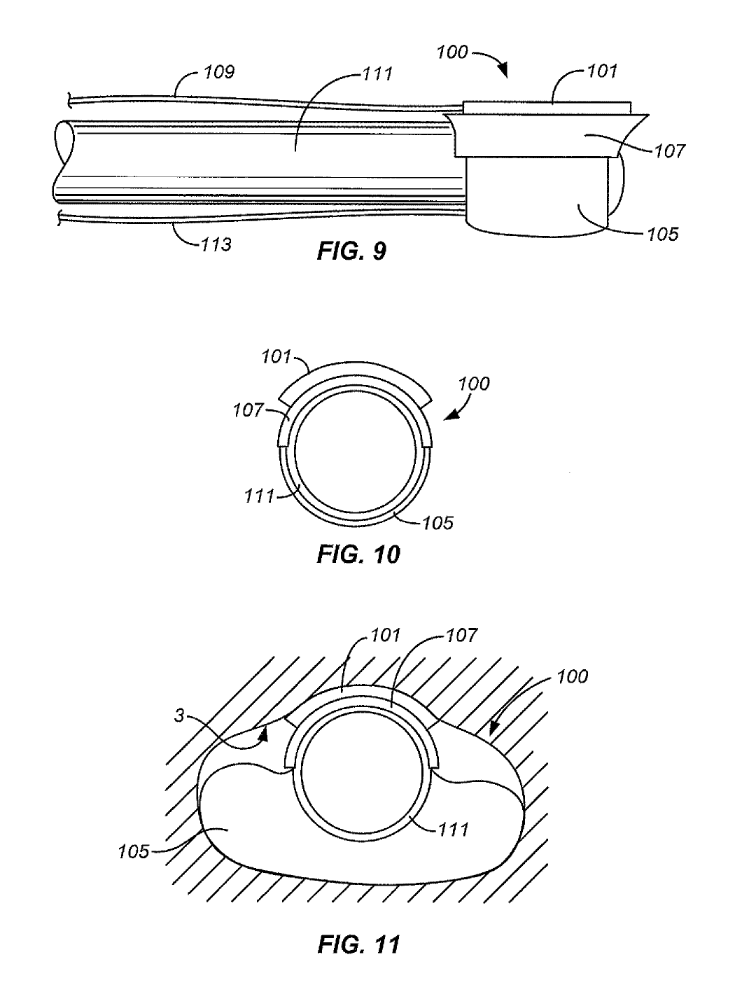

Additional and alternative device embodiments are included as described below, and depicted in FIGS. 9-23, 26-47, and 56. These device embodiments provide an ablational surface of less than a fully circumferential span. In terms of the circumference with respect to the device itself, some embodiments provide an ablational surface of about 90 degrees, some embodiments provide an ablational surface of about 180 degrees, however embodiments include any partially-circumferential span. Ablational energy elements include radiofrequency electrodes, among others, and may be arranged on the surface in any pattern, including fractionally-ablating patterns. The arc of a curved treatment area can be anything less than 360 degrees, however it is typically less than 180 degrees, and more particularly may include a smaller radial expanse such as arcs of about 5 degrees, about 10 degrees, about 15 degrees, about 30 degrees, about 45 degrees, about 60 degrees, and about 90 degrees.

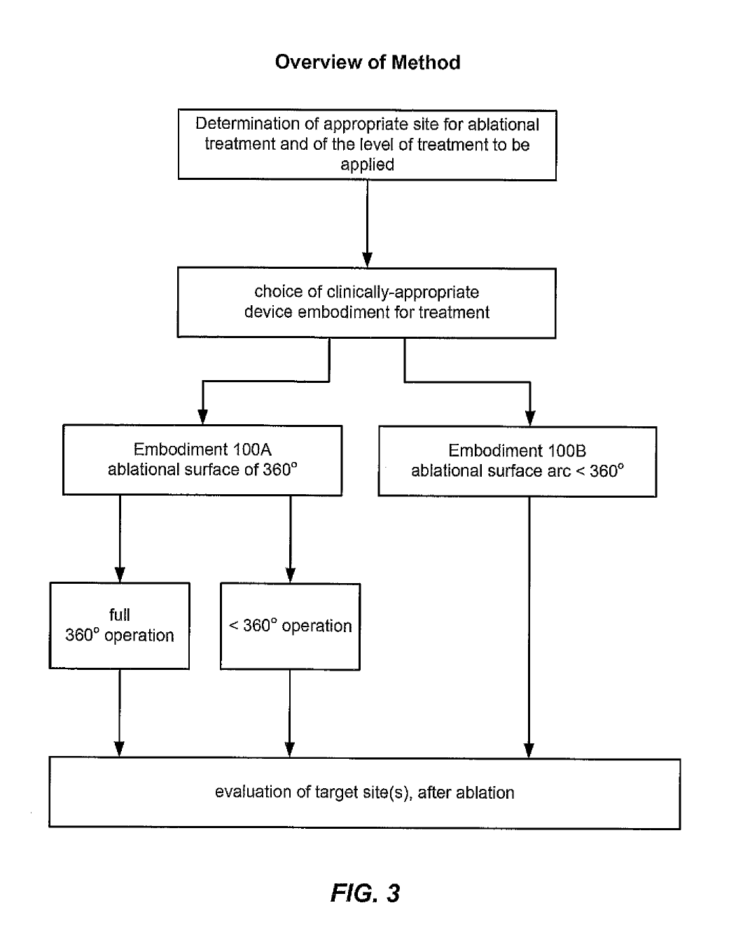

Turning now to an aspect of therapeutic ablation methods provided herein, that of determining an appropriate site for ablational treatment (FIG. 3), as well as the amount of ablational energy to be applied during such treatment, such determinations follow from the total amount of clinical information that a clinician can gather on a particular patient.

In some embodiments, a preliminary endoscopic or direct visual examination of the features of a lesion to be treated may be appropriate so that any patient-specific features may be mapped out, as well as an evaluation of the general dimensions of the patient's alimentary canal, particularly with regard to the specific anatomical location of the lesion. Such information may be obtained by direct visual observation or through an instrument such as an endoscope or colonscope. Still further, identification and/or localization of the lesion(s) may be accomplished by other diagnostic methods, including non-invasive penetrative imaging approaches such as narrow band imaging from an endoscope. In one aspect, evaluation of a site includes identifying the locale of the site, including size, orientation and dimensions of one or more lesions. In another aspect, evaluation of target tissue includes identifying a multiplicity of sites, if there is more than one site, and further identifying their locale and their respective dimensions. In still another aspect, evaluating target sites may include identifying or grading any pathology or injury to a specific site, particularly identifying any areas of clinical significance or concern that are overlapping or near the areas to be targeted for ablation.

Once target sites for ablation have been identified, target tissue containing the lesion may be treated with embodiments of an inventive ablational device and associated methods as described herein. Evaluation of the status of target tissue sites for ablation, particularly by visualization approaches, may also be advantageously implemented as part of an ablational therapy method (FIG. 3), as for example, in close concert with the ablation, either immediately before the application of ablational energy (such as radiant energy), and/or immediately thereafter. Further, the treatment site can be evaluated by any diagnostic or visual method at some clinically appropriate time after the ablation treatment, as for example a few days, several weeks, or several few months, or at anytime when clinically indicated following ablational therapy. In the event that any follow-up evaluation shows either that the therapy was unsatisfactorily complete, or that there is a recovery in the population of cells targeted for ablation, a repetition of the ablational therapy may be indicated.

Turning now to aspects of ablational devices that can be directed toward ablation based treatment of lesions, as described in detail herein, ablational devices have an ablational structure arrayed with energy-transmitting elements such as electrodes. In some embodiments, depending on the type of ablatative energy being used in the therapy, the devices may be mounted on, or supported by any appropriate instrument that allows movement of the ablational surface to the local of a target site. Such instruments are adapted in form and dimension to be appropriate for reaching the target tissue site, and may include simple catheters adapted for the purpose; some embodiments of the insertive instrument include endoscopes that, in addition to their supportive role, also provide a visualization capability. In some embodiments of the method, an endoscope separate from the supportive instrument may participate in the ablational procedure by providing visual information.

Exemplary embodiments of the inventive device as described herein typically make use of electrodes to transmit radiofrequency energy, but this form of energy transmission is non-limiting, as other forms of energy, and other forms of energy-transmission hardware are included as embodiments of the invention. Ablational energy, as provided by embodiments of the invention, may include, by way of example, microwave energy emanating from an antenna, light energy emanating from photonic elements, thermal energy transmitted conductively from heated ablational structure surfaces or as conveyed directly to tissue by heated gas or liquid, or a heat-sink draw of energy, as provided by cryonic or cryogenic cooling of ablational structure surfaces, or as applied by direct contact of cold gas or fluid with tissue, or by heat-draw through a wall of a device that separates the cold gas or fluid from the tissue.

Embodiments of the ablational device include variations with regard to the circumferential expanse of the ablational surface to be treated, some embodiments provide a fully circumferential ablation surface and others provide a surface that is less than fully circumferential, as described above. Choosing the appropriate device is a step included within the therapeutic method provided, as shown in FIG. 3. These and other variation may provide particular advantages depending on the nature, extent, locale, and dimensions of the one or more targeted tissue sites on the wall the alimentary canal. One embodiment of the invention includes a device with an ablational surface that is fully circumferential, i.e., encompassing a radius of 360 degrees, such that a full radial zone within a luminal organ is subject to ablation. Within that zone, ablation may be implemented to a varying degree, depending on the energy output and the pattern of the ablational elements (such as electrodes), but with substantial uniformity within the zone of ablation. This embodiment may be particularly appropriate for treating widespread or diffuse lesion sites. In another embodiment of the device, the ablational surface of the inventive device is partially circumferential, such that it engages a fraction of the full internal perimeter or circumference of a luminal organ. The fractional portion of the circumference ablated on the inner surface of a luminal organ depends on the size of the luminal organ being treated (radius, diameter, or circumference) and on the dimensions of the ablational surface, as detailed further below. With regard to treating target lesion sites that are small and discrete, the smaller or more discrete ablational surface provided by this latter embodiment may be advantageous. In some embodiments, the size of the ablation device corresponds to the size of the lesion or lesion site to be treated. In one aspect, the ablation device is selected to correspond to a size that is the same as the target lesion. In another aspect, the ablation device is selected to correspond to a size that is larger than the size of the target lesion.

This type of operational control of a circumferential subset of ablation energy elements around a 360-degree circumferential array is analogous to the fractional operation of a patterned subset of an electrode array, as described below in the section titled “Electrode patterns and control of ablation patterns across the surface area of tissue”. In the partially-circumferential operation of an array, a particular arc of the array is activated to deliver energy to an arc of the circumference. In the fractional-pattern operation of an array, energy is delivery to a portion of the tissue in the target area, while another portion receives insufficient energy to achieve ablation. In some embodiments, these operational variations can be combined, that is, a patterned subset of a circumferential arc can be activated.

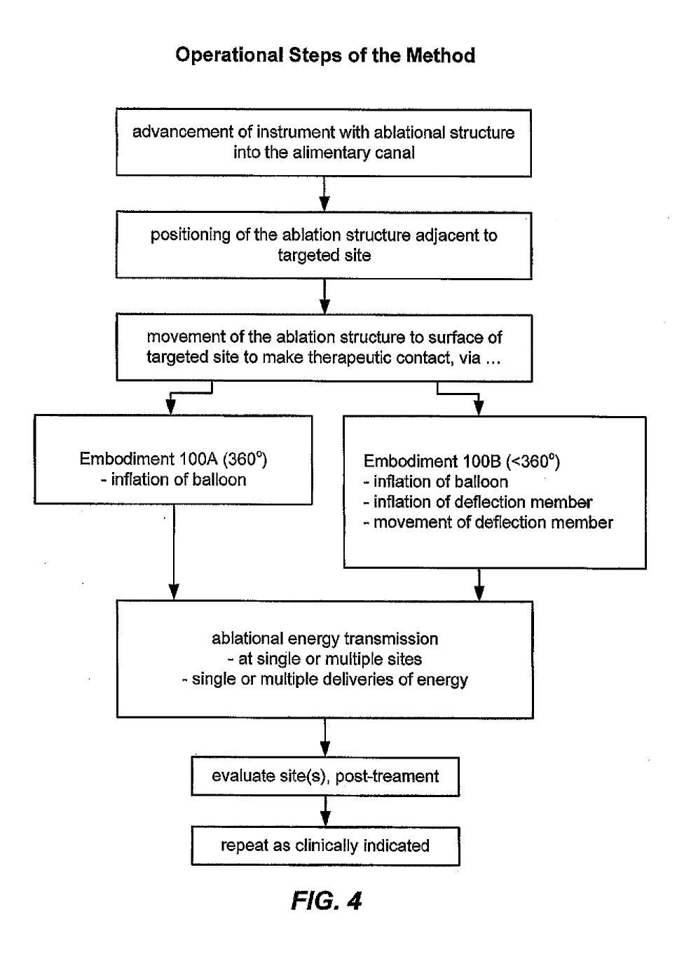

FIGS. 3 and 4 together provide flow diagram depictions of embodiments of the method for ablating tissue including a targeted lesion. The diagrams represent common aspects of the embodiments of the method, as delivered by two embodiments of the device, one which has a 360 degree circumferential ablation structure, and one which has an ablation structure comprising an arc of less than 360 degrees.

FIG. 3 is a flow diagram depicting an overview of the method with a focus on patient evaluation and determination of a clinically appropriate site within the alimentary canal for ablational treatment of a targeted lesion. In another step, a responsible clinician makes an informed choice with regard to the appropriate embodiment with which to treat the patient, i.e., either a device with the 360 degree electrode array 100A, or a device 100B with the electrodes arrayed in an arc of less than 360 degrees. In the event that the device 100A is chosen for use, another treatment choice may be made between operating the electrodes throughout the 360 degree circumference, or whether to operate a radial subset of the electrode array. In another step, a clinician further considers and makes a determination as to the protocol for ablation, considering the amount of energy to be delivered, the energy density, the duration of time over which energy is to be delivered. These considerations take into the account the surface area to be ablated, the depth of tissue which is to be treated, and the features of the electrode array, whether, for example, it is to be a fractional electrode, and which pattern may be desirable. Regardless of the device chosen, another preliminary step to operating the method may include a closer evaluation of the target tissue site(s) within the alimentary canal. Evaluation of the site may include the performance of any visualization or diagnostic method that provides a detailed census of the number of discrete target tissue sites, their dimensions, their precise locations, and/or their clinical status, whether apparently normal or abnormal. This step is shown following the choice of instrument, but may occur simply in conjunction with diagnosis, or at any point after diagnosis and general localization of the target tissue. In any case, an evaluating step is typically performed prior to ablation, as outlined in the operational steps of the method, as shown in the flow diagram of FIG. 4.

FIG. 4 is a flow diagram depicting an exemplary method of ablating a lesion once localized and a choice has been made regarding the preferred ablational device. The method includes an evaluation of the site, including lesion particulars such as location, stage, determination of the number of lesion sites, and the dimensions, as described above, and using approaches detailed in the references provided in the background, and/or by using whatever further approaches may be known by those practiced in the art. The method continues with insertion of the instrument and the movement of the ablational structure to the locale of the target tissue to be ablated. Subsequently, more refined movements of the ablational structure may be performed that create a therapeutically effective contact between the ablational structure and the target tissue site. In the event that the 360 degree embodiment of the device 100A is chosen, therapeutically effective contact may be made by inflating a balloon underlying the electrode array. In the event that the embodiment chosen is 100B, the device with an electrode surface spanning an arc of less than 360 degrees, movements that bring the ablational surface into therapeutically effective contact may include any of inflation of a balloon, inflation of a deflection member, and/or movement of a deflection member, all of which are described further below. The instrument or other device may also be used to deflect tissue in order to expose a target site containing a lesion.

After therapeutically-effective contact is made, by either device embodiment 100A or 100B, and by whatever type of movement was that was taken, a subsequent step includes the emission of ablational energy from the device. Variations of ablational energy emission may include ablating a single site as well as moving the instrument to a second or to subsequent sites that were identified during the evaluation step. Following the ablational event, a subsequent step may include an evaluation of the treated target site; alternatively evaluation of the consequences of ablation may include the gathering of clinical data and observation of the patient. In the event that an endoscope is included in the procedure, either as the instrument supporting the ablational structure, or as a separate instrument, such evaluation may occur immediately or very soon after ablation, during the procedure, when instruments are already in place. In other embodiments of the method, the treated site may be evaluated at any clinically appropriate time after the procedure, as for example the following day, or the following week, or many months thereafter. In the event that any of these evaluations show an ablation that was only partially complete, or show an undesired repopulation of targeted cells, the method appropriately includes a repetition of the steps just described and schematically depicted in FIG. 4.

In addition to observation by direct visual approaches, or other diagnostic approaches of site of ablation per se, evaluation of the consequences of ablation may include the gathering of a complete spectrum of clinical and metabolic data from the patient. Such information includes any test that delivers information relevant to the metabolic status of the patient such as the information gathered when determining the appropriateness of ablational intervention, as was made in the first step of FIG. 3.

### Device and Method for 360 Degree Circumferential Ablation

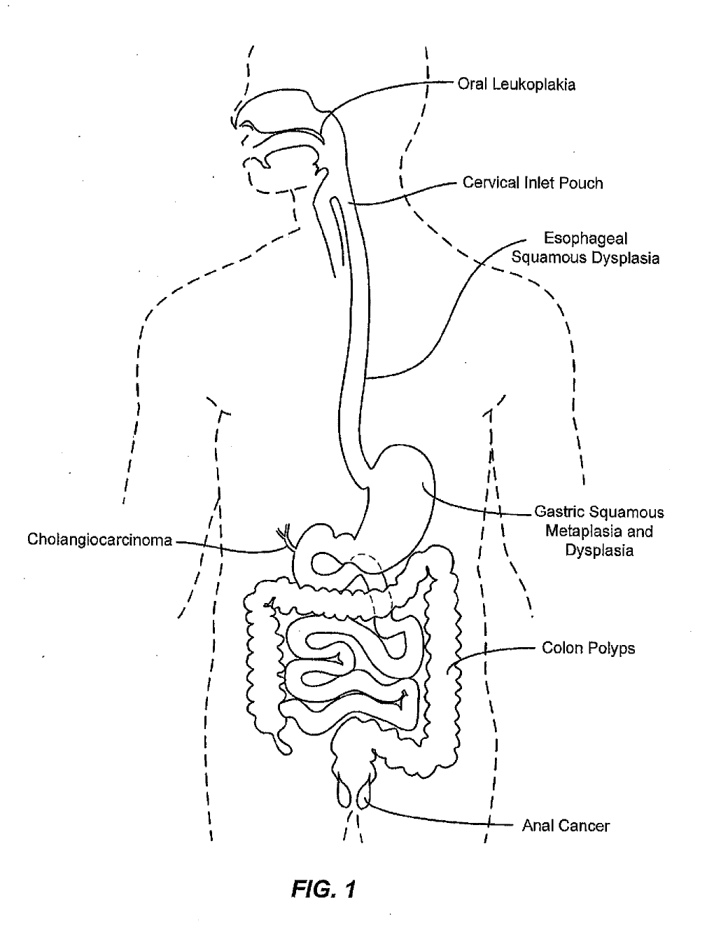

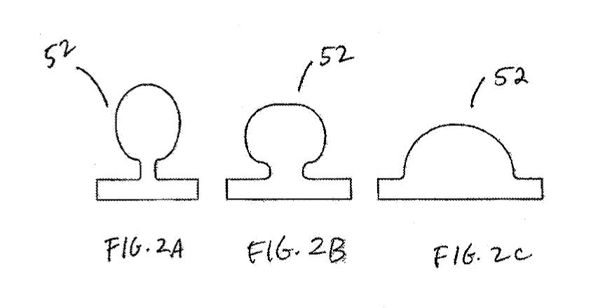

Methods for accomplishing ablation of targeted cells of a lesion according to this invention include the emission of radiant energy at conventional levels to accomplish ablation of the targeted lesion. In one embodiment, as shown in FIGS. 1A, 1C, and 2A, an elongated flexible shaft 41 is provided for insertion into the body in any of various ways selected by a medical care provider. The shaft may be placed endoscopically, e.g. passing through the mouth further into the proximal esophagus or other lesion site in the esophagus or the stomach. Alternatively, it may be placed surgically, or by any other suitable approach such as through or into the anus or rectum, as needed to access the lesion site.

In this embodiment, radiant energy distribution elements or electrodes on an ablation structure 101 are provided at a distal end of the flexible shaft 41 to provide appropriate energy for ablation as desired. In typical embodiments described in this section, the radiant energy distribution elements are configured circumferentially around 360 degrees. Alternatively to using emission of RF energy from the ablation structure, alternative energy sources can be used with the ablation structure to achieve tissue ablation and may not require electrodes. Such energy sources include: ultraviolet light, microwave energy, ultrasound energy, thermal energy transmitted from a heated fluid medium, thermal energy transmitted from heated element(s), heated gas such as steam heating the ablation structure or directly heating the tissue through steam-tissue contact, light energy either collimated or non-collimated, cryogenic energy transmitted by cooled fluid or gas in or about the ablation structure or directly cooling the tissue through cryogenic fluid/gas-tissue contact. Embodiments of the system and method that make use of these aforementioned forms of ablational energy include modifications such that structures, control systems, power supply systems, and all other ancillary supportive systems and methods are appropriate for the type of ablational energy being delivered.

In some embodiments of a fully circumferential ablation device, the flexible shaft comprises a cable surrounded by an electrical insulation layer and comprises a radiant energy distribution elements located at its distal end. In one form of the invention, a positioning and distending device around the distal end of the instrument is of sufficient size to contact and expand the walls of the gastrointestinal tract lumen or organ in which it is placed both in the front of the energy distribution elements as well as on the sides of the energy distribution elements. For example, the distal head of the instrument can be supported at a controlled distance from the wall of the gastrointestinal tract lumen or organ by an expandable balloon or inflation member, such that a therapeutically-effective contact is made between the ablation structure and the target site so as to allow regulation and control the amount of energy transferred to the target tissue within the lumen when energy is applied through the electrodes. The balloon is preferably bonded to a portion of the flexible shaft at a point spaced from the distal head elements.

Some embodiments of a fully-circumferential ablation device include a distendible or expandable balloon member as the vehicle to deliver the ablation energy. One feature of this embodiment includes means by which the energy is transferred from the distal head portion of the invention to the membrane comprising the balloon member. For example, one type of energy distribution that may be appropriate and is incorporated herein in its entirety is shown in U.S. Pat. No. 5,713,942, in which an expandable balloon is connected to a power source that provides radio frequency power having the desired characteristics to selectively heat the target tissue to a desired temperature. A balloon per embodiments of the current invention may be constructed of an electroconductive elastomer such as a mixture of polymer, elastomer, and electroconductive particles, or it may comprise a nonextensible bladder having a shape and a size in its fully expanded form which will extend in an appropriate way to the tissue to be contacted. In another embodiment, an electroconductive member may be formed from an electroconductive elastomer wherein an electroconductive material such as copper is deposited onto a surface and an electrode pattern is etched into the material and then the electroconductive member is attached to the outer surface of the balloon member. In one embodiment, the electroconductive member, e.g. the balloon member 105, has a configuration expandable in the shape to conform to the dimensions of the expanded (not collapsed) inner lumen of the human lower gastrointestinal tract.

In addition, such electroconductive member may consist of a plurality of electrode segments arrayed on an ablation structure 101 having one or more thermistor elements associated with each electrode segment by which the temperature from each of a plurality of segments is monitored and controlled by feedback arrangement. In another embodiment, it is possible that the electroconductive member may have means for permitting transmission of microwave energy to the ablation site. In yet another embodiment, the distending or expandable balloon member may have means for carrying or transmitting a heatable fluid within one or more portions of the member so that the thermal energy of the heatable fluid may be used as the ablation energy source.

Some embodiments of a fully circumferential ablation device include a steerable and directional control means, a means for accurately sensing depth of cautery, and appropriate alternate embodiments so that in the event of a desire not to place the electroconductive elements within the membrane forming the expandable balloon member it is still possible to utilize the balloon member for placement and location control while maintaining the energy discharge means at a location within the volume of the expanded balloon member, such as at a distal energy distribution head.

One approach a practitioner may use to determine the appropriate diameter ablation catheter to use with a particular patient is to use in a first step a highly compliant balloon connected to a pressure sensing mechanism. The balloon may be inserted into a luminal organ containing the target lesion and positioned at the desired site of the ablation and inflated until an appropriate pressure reading is obtained. The diameter of the inflated balloon may be determined and an ablation device of the invention having a balloon member capable of expanding to that diameter chosen for use in the treatment. In one aspect of the method of this invention, it is desirable to expand the expandable electroconductive member such as a balloon sufficiently to occlude the vasculature of the submucosa, including the arterial, capillary or venular vessels. The pressure to be exerted to do so should therefore be greater than the pressure exerted by such vessels.

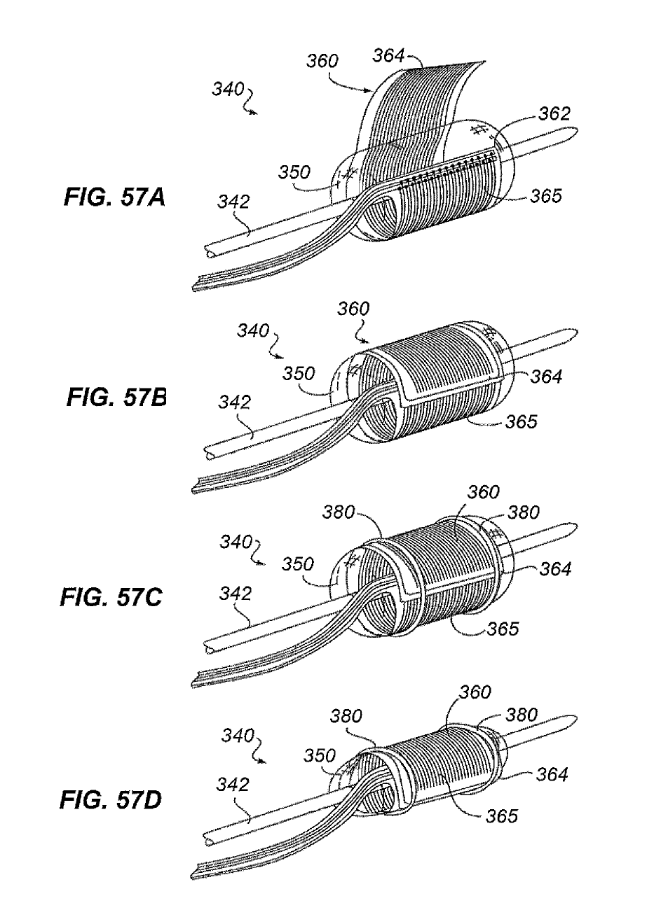

In other embodiments of the method, electronic means are used for measuring the luminal target area of the target lesion site so that energy may be appropriately normalized for the surface area of the target tissue. These aspects of the method are described in detail in U.S. patent application Ser. No. 12/143,404, of Wallace et al., entitled “Electrical means to normalize ablational energy transmission to a luminal tissue surface of varying size”, as filed on Jun. 20, 2008, which is incorporated in entirety. An embodiment of a device with a 360 degree ablational surface is described in detail in that application, and is depicted in FIGS. 57A-57D of this application. Pressure sensing means may also be used to measure the size of a lumen in preparation for an ablation treatment, as described in U.S. patent application Ser. No. 11/244,385 of Jackson, published as US 2006/0095032.

An embodiment of a device disclosed in U.S. patent application Ser. No. 12/143,404, of Wallace et al will be described here briefly, in order to provide an embodiment that includes a 360-degree ablational surface arranged on an overlapping support that expands in accordance with a balloon enclosed within the circumference of the support. Although the circumference of the device as a whole expands with the balloon, the ablational surface itself is non-distensible, and maintains its electrode density. FIGS. 57A-57D provide perspective views of an ablation device 100 with an overlapping electrode support 360 furled around an expandable balloon 105. An array of ablational energy delivery elements 101 such as radiofrequency electrodes is arranged on the exterior surface of the electrode support. The operative element is mounted on the distal end of an ablation catheter, of which the distal portion of a shaft 41 is seen, and around which the balloon 105 is configured. FIG. 57A shows the electrode support 360 pulled away from the balloon 105 to clarify that a portion of the support and an inner edge 362 is adherent to the balloon, and another portion and its outer edge 364 is not connected to the balloon. FIG. 57B shows the non-adherent portion of the electrode support 360 furled around the balloon 105 in a deployable configuration, the non-adherent portion and its edge overlapping around the adherent portion. FIG. 57C shows an optional feature of the device 100A, one or more elastic bands 380 wrapped around the electrode support 360. In some embodiments, the elastic band 380 material is a conductive elastomer, as described in greater detail below, which can be included in a size-sensing circuit to provide information related to the degree of expansion of the operative element. FIG. 57D shows the device of FIG. 57C in a collapsed state, with balloon portion 105 being uninflated (or deflated), this being the state of the device when it is being deployed into a lumen and being positioned at a target site, as well as the state of the device after delivering ablation energy and about to be removed from the lumen.

Another embodiment of an ablation device with a fully circumferential ablation surface is provided in FIGS. 58A-58B. This particular device embodiment 400 is adapted to present an ablational surface 101 into a concave or inwardly tapered target site such as distal portion of the antrum of the stomach, or in the vicinity of the pylorus. The device includes an ablational surface circumferentially arranged on the distal portion of an expandable member 105, the expandable member mounted around the distal end 110 of the shaft of an endoscope 111. FIG. 58A shows the device in a deployed configuration. FIG. 58B shows the device with the expandable member in an unexpanded or collapsed state, as would be appropriate for deployment of the device to a target tapered surface, or as would be appropriate for removal from the ablational site. FIG. 58C shows the device of FIG. 58A as it can be deployed into a tapered or concave target site such as the pylorus 9 or other portions of the stomach. FIG. 58D shows the device of FIG. 58A in an alternative configuration, with the electrode bearing surface of the device reversed such that it is facing proximally, and can thus be pulled retrograde into a tapered or concave site.

### Electrode Patterns and Control of Ablation Patterns Across the Surface Area of Tissue

Some aspects of embodiments of the ablational device and methods of use will now be described with particular attention to the electrode patterns present on the ablation structure. The device used is shown schematically in FIGS. 5-7. As shown in FIG. 6, the elongated flexible shaft 41 of a device with a fully circumferential ablation surface is connected to a multi-pin electrical connector 94 which is connected to the power source and includes a male luer connector 96 for attachment to a fluid source useful in expanding the expandable member. The elongated flexible shaft has an electrode 98 wrapped around the circumference. The expandable member of the device shown in FIGS. 5 and 6 further includes three different electrode patterns, the patterns of which are represented in greater detail in FIGS. 7A-7C. Typically, only one electrode pattern is used in a device of this invention, although more than one may be included. In the device shown in FIG. 5, the elongated flexible shaft 41 comprises six bipolar rings 62 with about 2 mm separation at one end of the shaft (one electrode pattern), adjacent to the bipolar rings is a section of six monopolar bands or rectangles 65 with about 1 mm separation (a second electrode pattern), and another pattern of bipolar axial interlaced finger electrodes 68 is positioned at the other end of the shaft (a third electrode pattern). In this device, a null space 70 is positioned between the last of the monopolar bands and the bipolar axial electrodes. The catheter used in the study was prepared using a polyimide flat sheet of about 1 mil (0.001″) thickness coated with copper. The desired electrode patterns were then etched into the copper.

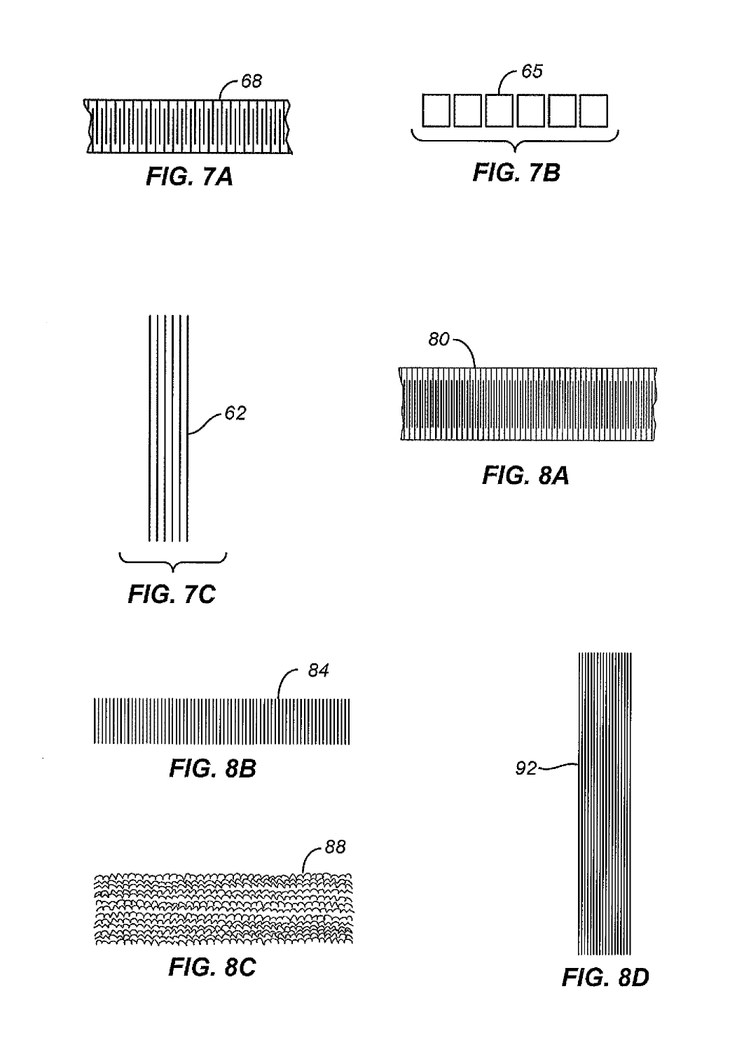

Alternative electrode patterns are shown in FIGS. 8A-8D as 80, 84, 88, and 92, respectively. Pattern 80 is a pattern of bipolar axial interlaced finger electrodes with about 0.3 mm separation. Pattern 84 includes monopolar bands with 0.3 mm separation. Pattern 88 is that of electrodes in a pattern of undulating electrodes with about 0.25 mm separation. Pattern 92 includes bipolar rings with about 0.3 mm separation. In this case the electrodes are attached to the outside surface of a balloon 72 having a diameter of about 18 mm. The device may be adapted to use radio frequency by attaching wires 74 as shown in FIG. 5 to the electrodes to connect them to the power source.

The preceding electrode array configurations are described in the context of an ablation structure with a full 360 degree ablation surface, but such patterns or variants thereof may also be adapted for ablation structures that provide energy delivery across a lesion target surface that is less than completely circumferential, in structures, for example, that ablate over any portion of a circumference that is less than 360 degrees, or for example structures that ablate around a radius of about 90 degrees, or about 180 degrees.

Embodiments of the ablation system provided herein are generally characterized as having an electrode pattern that is substantially flat on the surface of an ablation support structure and which is non-penetrating of the tissue that it ablates. The electrode pattern forms a contiguous treatment area that comprises some substantial radial aspect of a luminal organ; this area is distinguished from ablational patterns left by electrical filaments, filament sprays, or single wires. In some embodiments of the invention the radial portion may be fully circumferential; the radial portion of a luminal organ that is ablated by embodiments of the invention is function of the combination of (1) the circumference of the organ, (2) the dimensions of the electrode pattern and (3) the size and orientation of the target lesion site. Thus, at the high end, as noted, the radial expanse of a treatment area may be as large as 360 degrees, and as small as about 5 to 10 degrees, as could be the case in a treatment area within the stomach, the proximal esophagus, colon or anus

Embodiments of the ablational energy delivery system and method provided are also characterized by being non-penetrating of the target tissue. Ablational radiofrequency energy is delivered from the flat electrode pattern as it makes therapeutic contact with the tissue surface of a treatment area, as described elsewhere in this application; and from this point of surface contact, energy is directly inwardly to underlying tissue layers.

Some embodiments of the ablational system and method provided herein can be further characterized by the electrode pattern being configured to achieve a partial or fractional ablations, such that only a portion of the tissue surface receives sufficient radiofrequency energy to achieve ablation and another portion of the surfaces receives insufficient energy to achieve ablation. The system and method can be further configured to control the delivery of radiofrequency energy inwardly from the tissue surface such that depth of tissue layers to which energy sufficient for ablation is delivered is controlled.

Controlling the fraction of the tissue surface target area that is ablated, per embodiments of the invention, is provided by various exemplary approaches: for example, by (1) the physical configuration of electrode pattern spacing in a comparatively non-dense electrode pattern, and by (2) the fractional operation of a comparatively dense electrode array, in a billboard-like manner. Generally, creating a fractional ablation by physical configuration of the electrode pattern includes configuring the electrode pattern such that some of the spacing between electrodes is sufficiently close that the conveyance of a given level of energy between the electrodes sufficient to ablate tissue is allowed, and other spacing between electrodes is not sufficiently close enough to allow conveyance of the level of energy sufficient to ablate. Embodiments of exemplary electrode patterns that illustrate this approach to creating fractional ablation are described below, and depicted in FIGS. 48-55. The creation of an ablation pattern by activating a subset of electrodes represents an operation of the inventive system and method which is similar to the described above, wherein an ablational structure with a fully circumferential pattern of electrodes can be operated in a manner such that only a radial fraction of the electrodes are operated.

The ablation system of the invention includes an electrode pattern with a plurality of electrodes and a longitudinal support member supporting the electrode pattern, as described in numerous embodiments herein. Energy is delivered to the electrodes from a generator, and the operation of the generator is controlled by a computer-controller in communication with the generator, the computer controller controlling the operating parameters of the electrodes. The computer controller has the capability of directing the generator to deliver energy to all the electrodes or to a subset of the electrodes. The controller further has the ability to control the timing of energy delivery such that electrodes may be activated simultaneously, or in subsets, non-simultaneously. Further, as described elsewhere, the electrodes may be operated in a monopolar mode, in a bipolar mode, or in a multiplexing mode. These various operating approaches, particularly by way of activating subsets of electrodes within patterns, allow the formation of patterns that, when the pattern is in therapeutic contact with a target surface, can ablate a portion of tissue in the target area, and leave a portion of the tissue in the target area non-ablated.

Generally, creating a fractional ablation by an operational approach with a comparatively dense electrode array includes operating the electrode pattern such that the energy delivered between some of the electrodes is sufficient to ablate, whereas energy sufficient to ablate is not delivered between some of the electrodes. Embodiments of exemplary electrode patterns that illustrate this approach to creating fractional ablation are described below, and depicted in FIGS. 48-55.

Another aspect of controlling the fraction of tissue ablation, as well as controlled ablation generally relates to controlling the depth of ablation into lesion tissue layers within the target area. Energy is delivered inwardly from the surface, thus with modulated increases in energy delivery, the level of ablation can be controlled such that, for example, the ablated tissue may consist only of tissue in the epithelial layer. Additionally or alternatively, it may consist of tissue in the epithelial layer and the lamina propria layers, or it may consist of tissue in the epithelial, lamina propria and muscularis mucosal layers, or it may consist of tissue in the epithelial, lamina propria, muscularis mucosa, and submucosal layers, or it may consist of tissue in the epithelial layer, the lamina propria, the muscularis mucosae, the submucosa, and the muscularis propria layers. Alternatively, the depth of ablation into the layers of the targeted lesion or site may be controlled to ablate to a desired tissue layer.

Embodiments of the invention include RF electrode array patterns that ablate a fraction of tissue within a given single ablational area, exemplary fractional arrays are shown in FIGS. 48A, 49A, and 50A. These fractional ablation electrode arrays may be applied, as above, to above to ablational structures that address a fully circumferential target area, or a structure that addresses any portion of a full circumference such as 90 degree radial surface, or a 180 degree radial surface. FIG. 48A shows a pattern 180 of linear electrodes 60 aligned in parallel as stripes on a support surface. The electrodes are spaced apart sufficiently such that when pressed against tissue in therapeutic contact, the burn left by distribution of energy through the electrodes results in a striped pattern 190 on the target tissue as seen in FIG. 48B corresponding to the electrode pattern, with there being stripes of burned or ablated tissue 3a that alternate with stripes of unburned, or substantially unaffected tissue 3b. In some embodiments of the method, particularly in ablation structures that address a target area of less than 360 radial degrees, such as a target surface that is about 180 degrees, or more particularly about 90 degrees of the inner circumference of a lumen, the ablation may be repeated with the ablational structure positioned at a different angle. FIG. 48C, for example, depicts a tissue burn pattern 191 created by a first ablational event followed by a second ablational event after the ablational structure is laterally rotated by about 90 degrees. FIG. 48D, for another example, depicts a tissue burn pattern 192 created by a first ablational event followed by a second ablational event after the ablational structure is laterally rotated by about 45 degrees.

The effect of an ability to ablate a tissue surface in this manner adds another level of fine control over tissue ablation, beyond such parameters as total energy distributed, and depth of tissue ablation. The level of control provided by fractional ablation, and especially when coupled with repeat ablational events as described above in FIGS. 48C and 48D, is to modulate the surface area-distributed fraction of tissue that is ablated to whatever degree the local maximal ablation level may be. The fractional ablation provided by such fractional electrode pattern may be particularly advantageous when the effects of ablation are not intended to be absolute or complete, but instead a functional compromise of tissue, or of cells within the tissue is desired. In some therapeutic examples, thus, a desirable result could be a partial reduction in overall function of a target area, rather than a total loss of overall function.

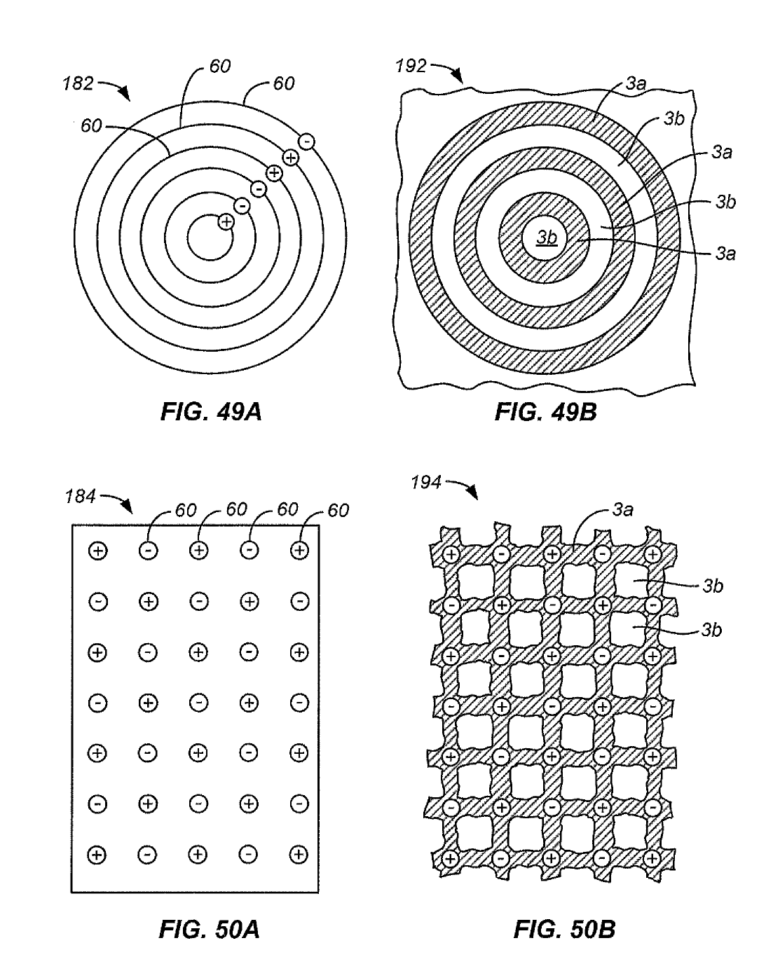

FIGS. 49A and 50A depict other examples of a fractionally-ablating electrode pattern on an ablation structure, and FIGS. 49B and 50B show the respective fractional burn patterns on tissue that have been treated with these electrode patterns. In FIG. 49A a pattern of concentric circles 182 is formed by wire electrodes that (from the center and moving outward) form a +−−++− pattern. When activated, the tissue between +− electrodes is burned, and the tissue between ++ electrode pairs or −− electrode pairs is not burned. Thus, the concentric pattern 192 of FIG. 49B is formed. Embodiments of fractionally-ablating electrode patterns such as those in FIG. 49A need not include perfect circles, and the circles (imperfect circles or ovals) need not be perfectly concentric around a common center.

Similarly, FIG. 50A shows a checkerboard pattern 184 of + and − electrodes which when activated create a burn pattern 194 as seen in FIG. 50B. Tissue that lies between adjacent + and − electrodes is burned, while tissue that lies between adjacent ++ electrodes or −− electrode pairs remains unburned. FIG. 50B includes a representation of the location of the + and − electrodes from the ablation structure in order to clarify the relative positions of areas that are burned 3a and the areas that remain substantially unburned 3b.

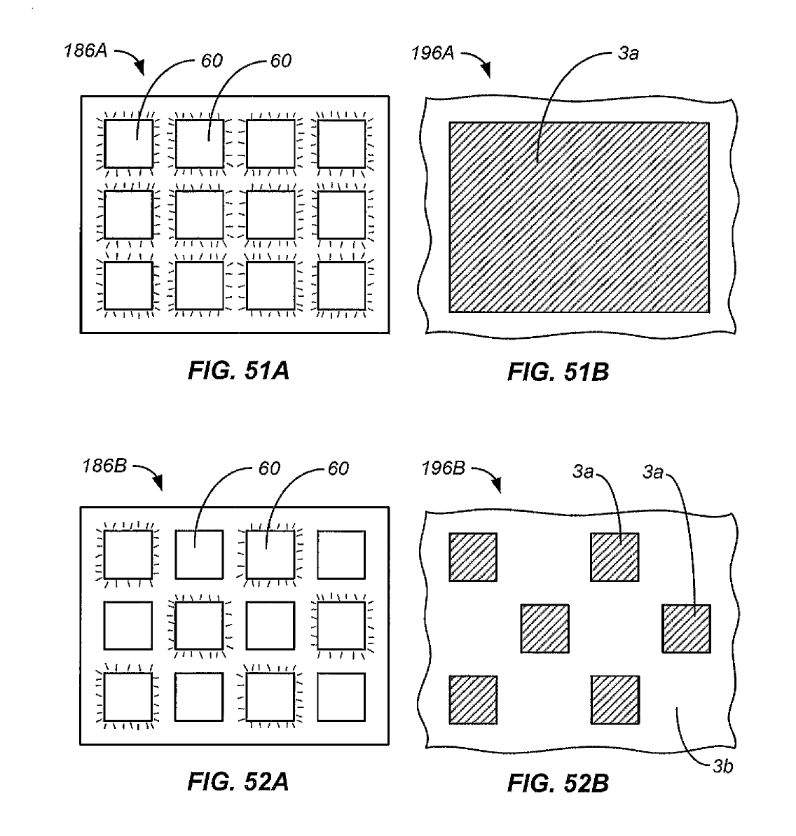

Embodiments of the invention include RF electrode array patterns that ablate a fraction of tissue within a given single ablational area by virtue of operational approaches, whereby some electrodes of a pattern are activated, and some are not, during an ablational event visited upon a target area. Exemplary fractional arrays are shown in FIGS. 51A, 52A, 53A and 54A. These fractional ablation electrode arrays may be applied, as above, to ablational structures that address a fully circumferential target area, or a structure that addresses any portion of a full circumference such as, by way of example, a 90 degree radial surface, or a 180 degree radial surface.

FIG. 51A shows a checkerboard electrode pattern during an ablational event during which all electrode squares of the operational pattern 186A are operating, as depicted by the sparkle lines surrounding each electrode. Operating the electrode pattern 186A in this manner produces an ablation pattern 196A, as seen in FIG. 51B, wherein the entire surface of tissue within the treatment area is ablated tissue 3a. FIG. 52A, on the other hand, shows a checkerboard electrode pattern during an ablational event during which only every-other electrode square of the operational pattern 186B is operating, as depicted by the sparkle lines surrounding each activated electrode. Operating the electrode pattern 186B in this manner produces an ablation pattern 196B, as seen in FIG. 52B, wherein a checkerboard fractionally ablated pattern with a dispersed pattern of ablated squares 3a of tissue 3a alternate with square areas of tissue 3b that are not ablated.

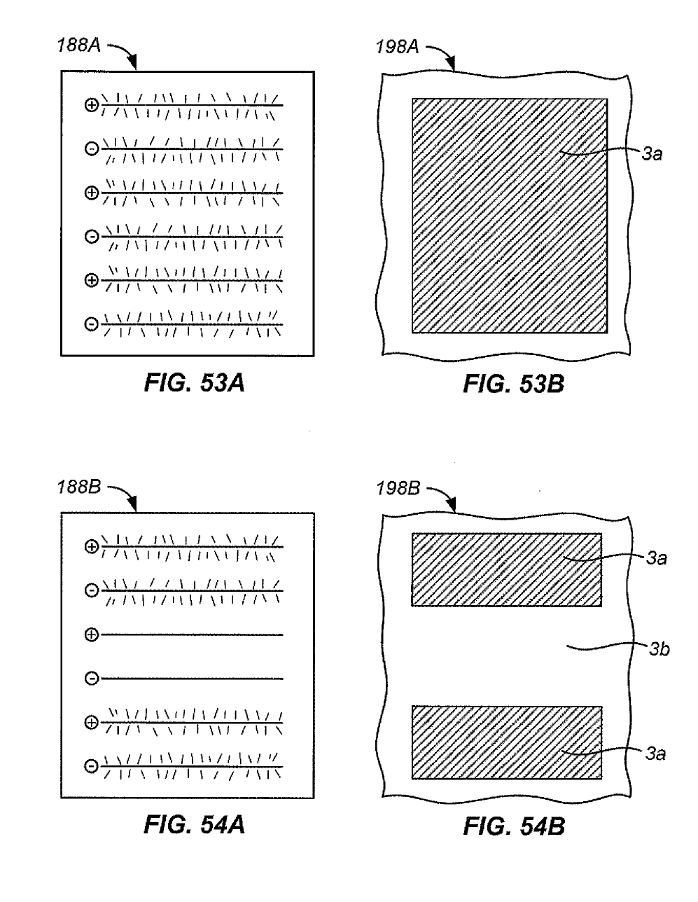

FIG. 53A shows a striped linear electrode pattern of alternating + and − electrodes during an ablational event during which all electrode squares of the operational pattern 188A are operating, as depicted by the sparkle lines surrounding each linear electrode. Operating the electrode pattern in this manner 188A produces an ablation pattern 198A, as seen in FIG. 53B, wherein the entire surface of tissue within the treatment area is ablated tissue 3a.

FIG. 54A, on the other hand, shows a striped linear electrode pattern 188B of alternating + and − electrodes during an ablational event during which alternate pairs of the linear electrode pairs are operating, as depicted by the sparkle lines surrounding the activated linear electrodes. Operating the electrode pattern in this manner 188B produces an ablation pattern 198B, as seen in FIG. 54B, wherein stripes of ablated tissue 3a within the treatment area alternate stripes of non-ablated tissue 3b.

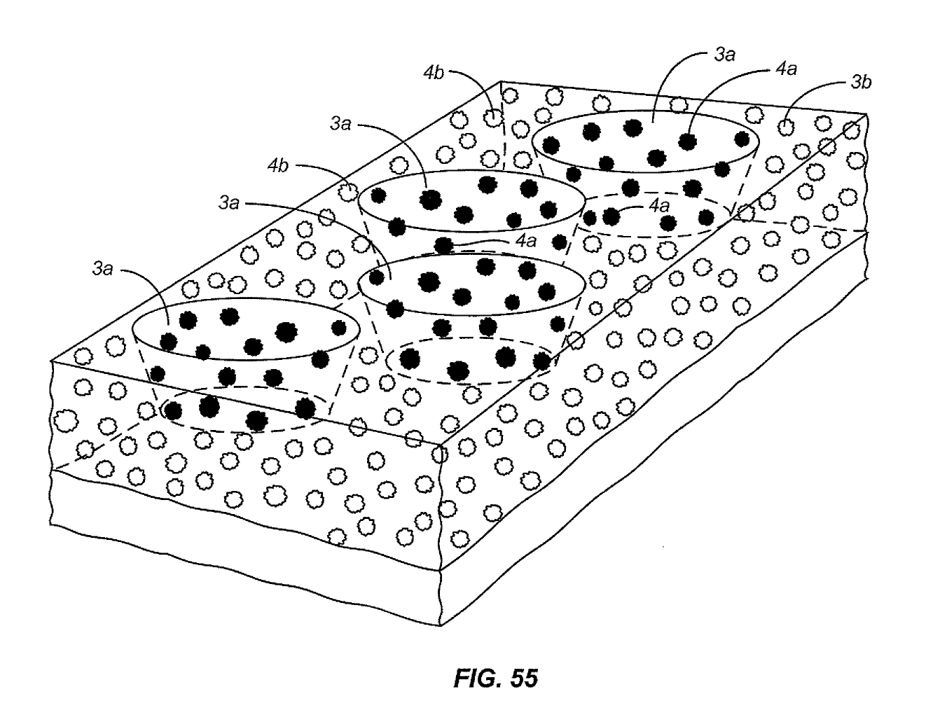

FIG. 55 is a schematic rendering of a three dimensional view of a target lesion region after it has been ablationally treated, per embodiments of the invention. Ablated regions 3a are rendered as regions distributed through the target area within a larger sea of non-ablated tissue 3b. These regions are depicted as being slightly conical in this schematic view, but in practice the ablated tissue region may be more cylindrical in shape. The regions 3a are of approximately the same depth, because of the control exerted over the depth of the ablation area into tissue layers, as described herein. With such control, the regions 3a can vary with respect of the layer to which they extend continuously from the upper surface where ablational energy has been applied. The conical regions are of approximately the same width or diameter, and distributed evenly throughout the tissue, because of the control over ablational surface area, as described herein. In this particular example, the therapeutic target is actually a particular type of cell 4b (open irregular spheres), for example, a nerve cell, or endocrine secretory cell; and these cells are distributed throughout the target area. The post-ablation therapeutic target cells 4a (dark irregular spheres) are those which happened to be included within the conical regions 3a that were ablated. The post-ablation cells 4a may be rendered dysfunctional to varying degree, they may be completely dysfunctional, they may be, merely by way of illustrative example, on the average, 50% functional by some measure, and there functionality may vary over a particular range. It should be particularly appreciated however, per embodiments of the invention, that the cells 4b, those not included in the ablated tissue cones, are fully functional.

### Controlling the Ablation in Terms of the Tissue Depth of the Ablation Effect

In addition to controlling the surface area distribution of ablation, as may be accomplished by the use of fractional ablation electrodes as described above, or as controlled by the surface area of electrode dimensions, ablation can be controlled with regard to the depth of the ablation below the level of the tissue surface where the ablative structure makes therapeutic contact with the tissue. The energy delivery parameters appropriate for delivering ablation that is controlled with regard to depth in tissue may be determined experimentally.

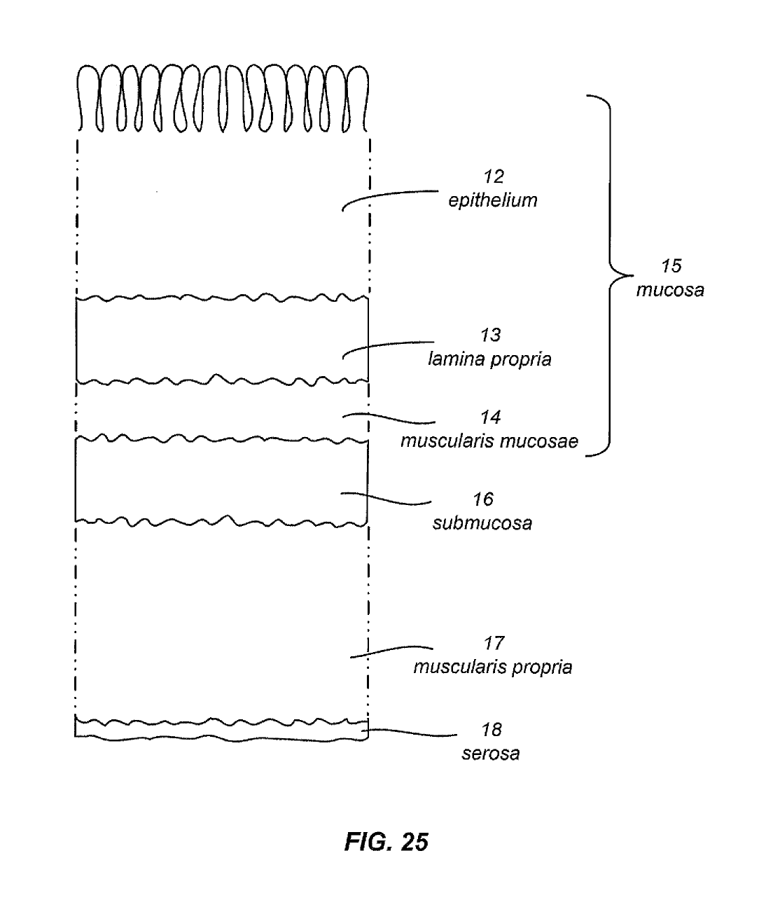

FIG. 25 provides a schematic representation of the histology of a target lesion wall as it is generally found within the alimentary canal. The relative presence and depth and composition of the layers depicted in FIG. 25 vary depending on anatomical position, but the basic organization is similar. The layers of the target site wall will be described in their order as seen FIG. 25 from top to bottom and in terms of the direction from which an ablation structure would approach the tissue. The innermost layer can be referred to as the surface (epithelium), and succeeding layers can be understood as being below or beneath the “upper” layers. The innermost layer of a target site within the gastrointestinal tract is in direct contact with the nutrients and processed nutrients as they move through the gut is a layer of epithelium 12. This layer secretes mucous which protects the lumen from abrasion and against the corrosive effect of an acidic environment. Beneath the epithelium is a layer known as the lamina propria 13, and beneath that, a layer known as the muscularis mucosae 14. The epithelium 12, the lamina propria 13, and the muscularis mucosae 14 collectively constitute the mucosa 15.

Below the mucosal layer 15 is a layer known as the submucosa 16, which forms a discrete boundary between the muscosal layer 15 above, and the muscularis propria 17 below. The muscularis propria 17 if present includes various distinct layers of smooth muscle that enwrap the organ, in various orientations, including oblique, circular, and the longitudinal layers. Enwrapping the muscularis propria 17 is the serosa 18, which marks the outer boundary of the organ.

As provided by embodiments of the invention, the ablation applied to lesion tissue may be depth-controlled, such that only the epithelium 12, or only a portion of the mucosal layer is ablated, leaving the deeper layers substantially unaffected. In other embodiments, the ablated tissue may commence at the epithelium yet extend deeper into the submucosa and possibly the muscularis propria, as necessary to achieve the desired therapeutic effect.

### Device and Method for Partially-Circumferential Ablation

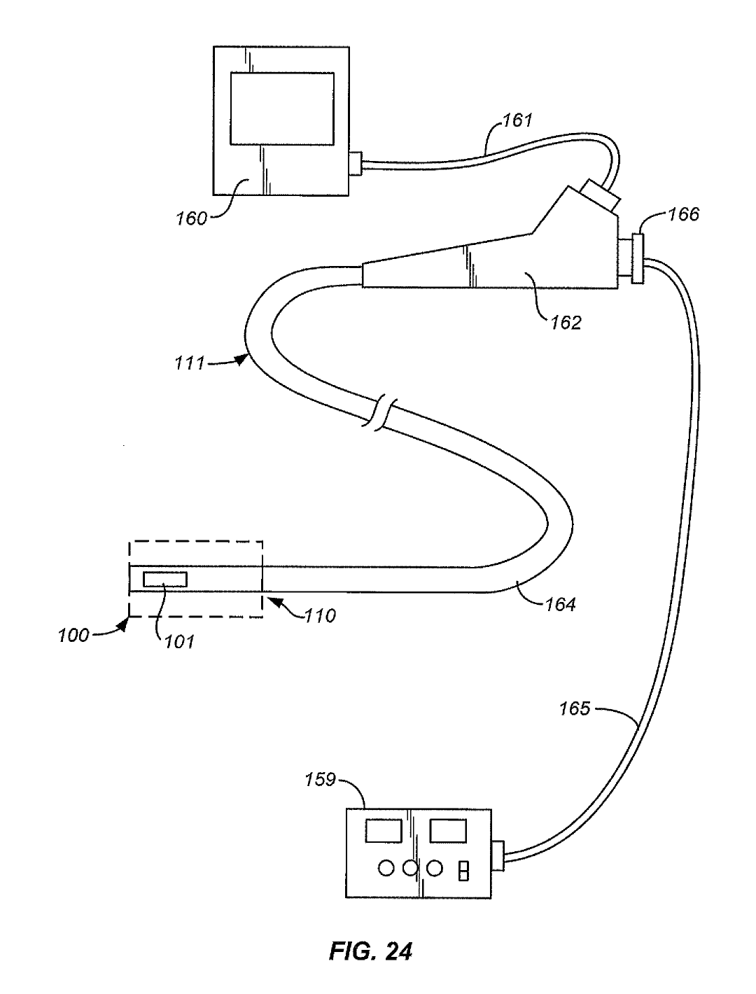

One embodiment of a method of ablating lesion tissue includes the use of an ablation device with an ablation structure supported by conventional endoscopes 111, as illustrated in FIG. 24. An example of one commercially available conventional endoscope 111 is the Olympus “gastrovideoscope” model number GIF-Q160. While the specific construction of particular commercially available endoscopes may vary, as shown in FIG. 24, most endoscopes include a shaft 164 having a steerable distal end 110 and a hub or handle 162 which includes a visual channel 161 for connecting to a video screen 160 and a port 166 providing access to an inner working channel within the shaft 164. Dials, levers, or other mechanisms (not shown) will usually be provided on the handle 162 to allow an operator to selectively steer the distal end 110 of the endoscope 111 as is well known in the endoscopic arts. In accordance with the present invention, an ablation device, including an ablation structure is advanced while supported at the distal end of an endoscope. The ablation structure is deflectable toward a tissue surface and the ablation structure is activated to ablate the tissue surface. Within the gastrointestinal tract, variously sized tissue surface sites can selectively be ablated using the device. As will be further described, the ablational structure of embodiments described in this section do not circumscribe a full 360 degrees, but rather circumscribe a fraction of 360 degrees, as will be described further below.

In general, in one aspect a method of ablating lesion tissue in the gastrointestinal tract is provided, more particularly lesions such as benign, pre-cancerous and early cancerous lesions that originate within the epithelium and are limited to the mucosal layer of the gastrointestinal tract. The method includes advancing an ablation structure into the gastrointestinal tract while supporting the ablation structure with an endoscope. In some embodiments, advancing the structure into the gastrointestinal tract may be sufficient to place the ablational structure of the device into close enough proximity in order to achieve therapeutic contact. In other embodiments, a subsequent step may be undertaken in order to achieve an appropriate level of therapeutic contact. This optional step will be generally be understood as moving the ablation structure toward the target lesion site. The method thus may further include moving at least part of the ablation structure with respect to the endoscope and toward a tissue surface; and activating the ablation structure to ablate the tissue surface. Moving at least part of the ablation structure with respect to the endoscope can include movement toward, away from or along the endoscope. Moving the ablational structure toward a target tissue surface may be performed by structures in ways particular to the structure. For example, the structure can be moved by inflating a balloon member, expanding a deflection member, or moving a deflection member. The function of such movement is to establish a therapeutically effective contact between the ablational structure and the target site. A therapeutically effective contact includes the contact being substantial and uniform such that the highly controlled electrical parameters of radiant emission from the electrode result in similarly highly controlled tissue ablation. Some embodiments of the invention further include structure and method for locking or securing such a therapeutically effective contact once established. Thus, some embodiments include a position locking step that, for example, uses suction to secure the connection between the ablation structure and the tissue site.

As shown in FIGS. 9, 10, 11, and 26, in one aspect a method of ablating lesion tissue includes an ablation device 100 for ablating a tissue surface 3, wherein the device 100 includes an ablating structure, for example, an ablation structure 101 supported by an endoscope 111. The method includes ablating tissue in a lesion region by the steps of (1) advancing the ablation structure 101 into the lesion region; (2) deflecting the ablation structure 101 toward a lesion tissue surface 3; and (3) activating the ablation structure to ablate the lesion 3. As shown in FIG. 9, the device 100 can additionally include a housing 107, electrical connections 109, an inflation line 113 and an inflation member or balloon 105.

The ablation structure 101, in one embodiment is an electrode structure configured and arranged to deliver energy comprising radiofrequency energy to the mucosal layer. It is envisioned that such an ablation structure 101 can include a plurality of electrodes. For example, two or more electrodes may be part of an ablation structure. The energy may be delivered at appropriate levels to accomplish ablation of mucosal or submucosal level tissue, or alternatively to cause therapeutic injury to these tissues, while substantially preserving muscularis tissue. The term “ablation” as used herein generally refers to thermal damage to the tissue causing any of loss of function that is characteristic of the tissue, or tissue necrosis. Thermal damage can be achieved through heating tissue or cooling tissue (i.e. freezing). In some embodiments ablation is designed to be a partial ablation.

Although radiofrequency energy, as provided by embodiments of the invention, is one particular form of energy for ablation, other embodiments may utilize other energy forms including, for example, microwave energy, or photonic or radiant sources such as infrared or ultraviolet light, the latter possibly in combination with improved sensitizing agents. Photonic sources can include semiconductor emitters, lasers, and other such sources. Light energy may be either collimated or non-collimated. Other embodiments of this invention may utilize heatable fluids, or, alternatively, a cooling medium, including such non-limiting examples as liquid nitrogen, Freon™, non-CFC refrigerants, CO₂ or N₂O as an ablation energy medium. For ablations using hot or cold fluids or gases, the ablation system may include an apparatus to circulate the heating/cool medium from outside the patient to the heating/cooling balloon or other element and then back outside the patient again. Mechanisms for circulating media in cryosurgical probes are well known in the ablation arts. For example, and incorporated by reference herein, suitable circulating mechanisms are disclosed in U.S. Pat. No. 6,182,666 to Dobak; U.S. Pat. No. 6,193,644 to Dobak; U.S. Pat. No. 6,237,355 to Li; and U.S. Pat. No. 6,572,610 to Kovalcheck.

In a particular embodiment, the energy delivered to the lesion in the gastrointestinal tract comprises radiofrequency energy that can be delivered from the energy delivery device 100. Radiofrequency energy can be delivered in a number of ways. Typically, the radiofrequency energy will be delivered in a bipolar fashion from a bipolar array of electrodes positioned on the ablation structure 101, in some cases on an expandable structure, such as a balloon, frame, cage, or the like, which can expand and deploy the electrodes directly against or immediately adjacent to the mucosal tissue so as to establish a controlled level of therapeutic contact between the electrodes and the target tissue (e.g., through direct contact or through a dielectric membrane or other layer). Alternatively, the electrode structure may include a monopolar electrode structure energized by a radiofrequency power supply in combination with a return electrode typically positioned on the patient's skin, for example, on the small of the back. In any case, the radiofrequency energy is typically delivered at a high energy flux over a very short period of time in order to injure or ablate only the mucosal or submucosal levels of tissue without substantially heating or otherwise damaging the muscularis tissue. In embodiments where the ablation structure includes a plurality of electrodes, one or more of the electrodes can be bipolar or monopolar, and some embodiments include combinations of bipolar and monopolar electrodes.

The ablation structure 101 can be arranged and configured in any of a number ways with regard to shape and size in order to treat the targeted lesion. Typically, the array has an area in the range from about 0.5 cm² to about 9.0 cm². Typical shapes would include rectangular, circular or oval. In one embodiment, the ablation structure 101 has an area of about 2.5 cm². In another embodiment, the ablation structure 101 has an area of about 4 cm² and dimensions of about 2 cm. by 2 cm.

The housing 107 of the ablation device 100 is arranged and configured to support the ablation structure 101. The housing 107 can be made of any suitable material for withstanding the high energy flux produced by the ablation structure 101. As shown in FIGS. 9-14, 17, 18, 21, and 22, in one embodiment, the housing 107 is sandwiched between the ablation structure 101 and an endoscope 111 when the ablation device 100 is supported by an endoscope 111. One end of the ablation structure 101 can be further away from the endoscope than the other end to improve ease of contact with the targeted tissue (not shown). For example, to ensure the proximal end of the ablation structure 101 makes contact with the targeted tissue, the proximal end of the electrode may be supported by a tapered housing member 107.

The electrical connections 109 of the ablation device connect the ablation structure 101 to a power source. The electrical connections 109 can include a single wire or plurality of wires as needed to provide controlled energy delivery through the ablation structure 101. In one embodiment, the electrical connections 109 include low electrical loss wires such as litz wire.

The inflation line 113 is arranged and configured to transport an expansion medium, typically a suitable fluid or gas, to and from the inflation member. In one embodiment, the inflation line is a flexible tube. The inflation line 113 can be made of polymer or co-polymers, such as the non-limiting examples of polyimide, polyurethane, polyethylene terephthalate (PET), or polyamides (nylon). The inflation member 105 is designed to deflect the ablation device 100 in relation to a target tissue surface 3. The inflation member 105 can be reversibly expanded to an increased profile.

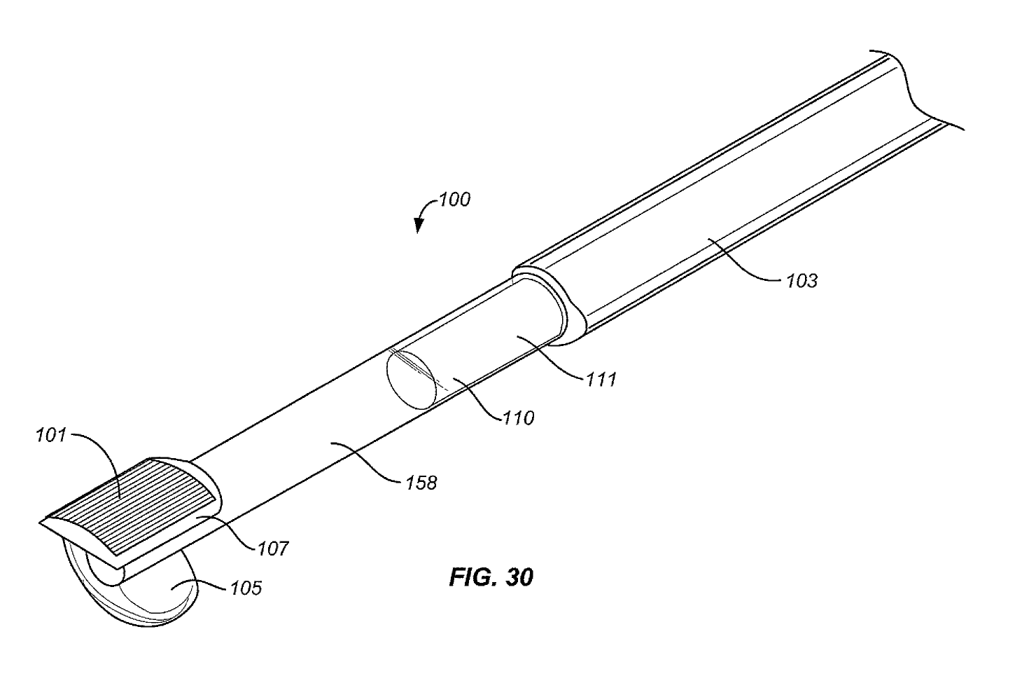

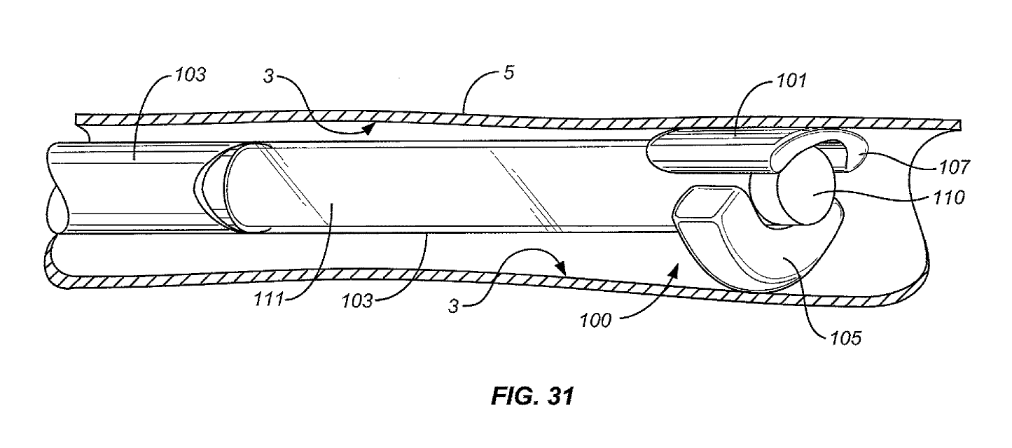

In one embodiment, the inflation member 105 additionally serves as an attachment site for support of the ablation device 100 by an endoscope 111. As shown in FIGS. 9-14, 17, 18, 21 and 22, the inflation member 105 can be deployed from a low profile configuration or arrangement (see FIGS. 10, and 20) to an increased profile configuration or arrangement (see FIGS. 11-14, 17-19) using the expansion medium. In preparation for ablation, when the inflation member 105 is sufficiently inflated, deflection of the ablation device 100 in relation to a tissue surface 3 can be achieved. As shown in FIGS. 11, 31, 42, and 44, in one embodiment, deflection of the ablation device 100 results in a therapeutic level of contact, i.e., a substantially direct, uniform, and sustainable contact between the ablation structure 101 of the device 100 and the target tissue surface 3. For example, as shown in FIGS. 31, 42, and 44, when the inflation member 105 is sufficiently inflated, the resulting expanded profile of the inflation member 105, which contacts the tissue surface 3, results in contact by deflection between the tissue surface 3 and the ablation structure 100. In these embodiments, suction can be applied in combination with the inflation member 105 to achieve contact between the ablation structure 101 and the tissue surface 3. Suction can be achieved through the endoscope 111 or through the ablation device 100 to aid in collapsing the targeted tissue surface 3 around the ablation structure 101.

In various embodiments, the inflation member 105 may be compliant, non-compliant or semi-compliant. The inflation member 105 can be made of a thin, flexible, bladder made of a material such as a polymer, as by way of non-limiting examples, polyimide, polyurethane, or polyethylene terephthalate (PET). In one embodiment, the inflation member is a balloon. Inflation of the inflation member 105 can be achieved through the inflation line 113 using, for example, controlled delivery of fluid or gas expansion medium. The expansion medium can include a compressible gaseous medium such as air. The expansion medium may alternatively comprise an incompressible fluid medium, such as water or a saline solution.

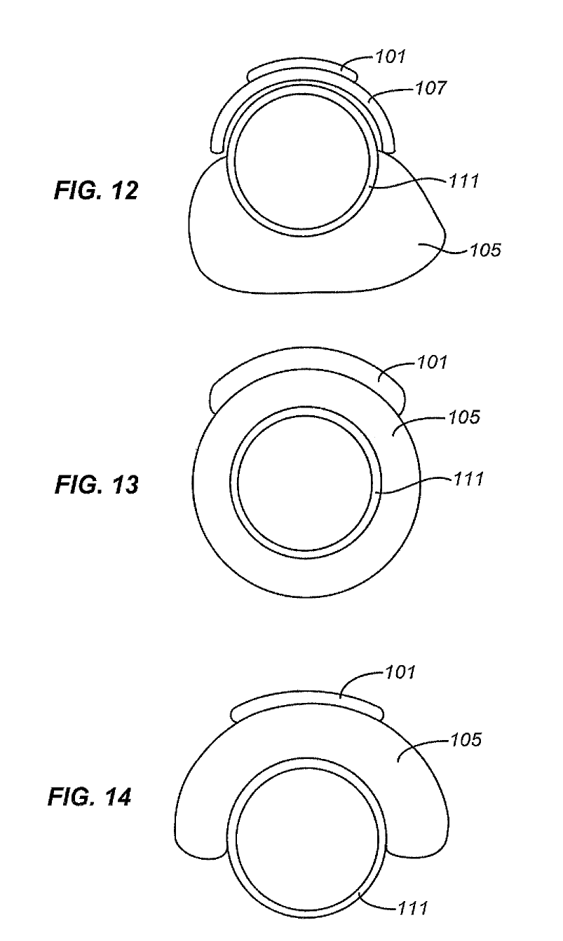

As shown in FIGS. 12, 13, and 14, the inflation member 105 can be configured and arranged in a variety of ways to facilitate deflection of the ablation device 100 in relation to a tissue surface 3. For example, as shown in FIG. 12, the inflation member 105 can be eccentrically positioned in relation to the supporting endoscope 111 as well as the housing 107 and the ablation structure 101. Alternatively, as shown in FIG. 13, the inflation member 105 can be positioned concentrically in relation to the supporting endoscope 111 and the ablation structure 101 can be attached to the inflation member 105 distally from the endoscope 111. In another embodiment, as shown in FIG. 12, the inflation member 105 can be positioned between the supporting endoscope 111 and the ablation structure 101. The ablation structure 101 shown in FIGS. 12-14 can cover a range of circumferential span of the endoscope 111 spanning, for example, from about 5 to 360 degrees when inflation member 105 is deployed.

One method of ablating tissue in a targeted lesion region can include a first step of advancing an ablation structure 101, into the targeted lesion region. Next, the ablation structure 101 is deflected toward a lesion tissue surface. Finally, energy can be applied to the ablation structure 101 to ablate the lesion.

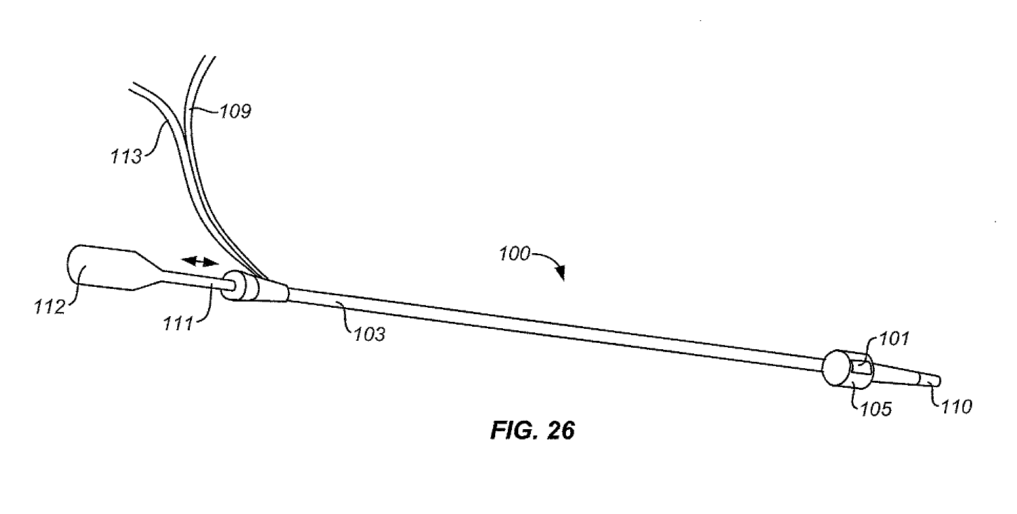

In a further method, the step of supporting the ablation structure 101 with an endoscope 111 includes inserting the endoscope 111 into the ablation structure 101 (see for example, FIGS. 1A-2B). In a related method, the ablation structure 101 is supported by a sheath 103 (see FIGS. 26-28, 30, 31, 32 and 37) and the step of inserting the endoscope 111 into the ablation structure 101 includes inserting the endoscope 111 into the sheath 103. In a further related method, the step of inserting the endoscope 111 into the sheath 103 includes creating an opening in the sheath 103 (not shown).

In a particular method, a distal portion of a sheath 103 having a smaller outer diameter than a proximal portion of the sheath 103, is adapted to be expanded when an endoscope 111 is inserted into it.

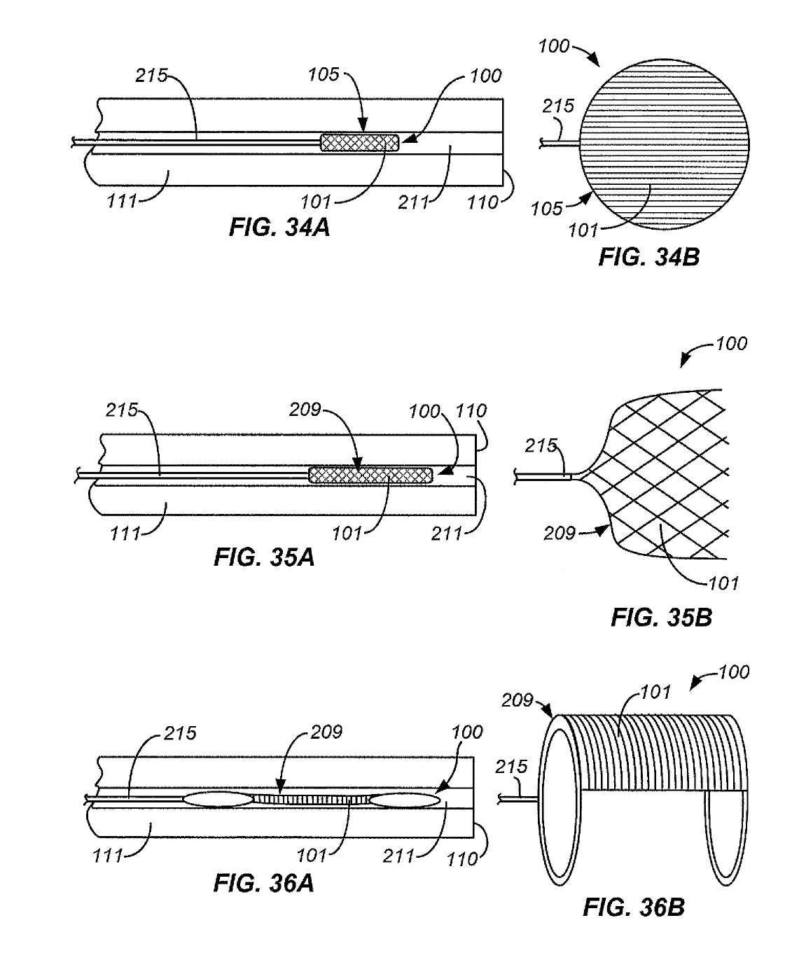

In another method, the step of advancing the ablation structure 101 into the gastrointestinal tract or lesion region includes advancing the ablation structure 101 through a channel of the endoscope 111 from either the endoscopes proximal or distal end (as discussed below for FIGS. 34A, 35A and 36A). In yet another method, the step of supporting the ablation structure 101 comprises supporting the ablation structure 101 with a channel of the endoscope (see as discussed below for FIGS. 34A, 35A, 36A, 37-39). In a further method, a deflection structure or deflection member 150 is advanced through a channel of the endoscope 111 and the step of deflecting the ablation structure 101 toward a tissue surface 3 includes deflecting the ablation structure 101 with the deflection structure or deflection member 150.

As illustrated in FIGS. 34A, 35A, and 36A, variously adapted and configured ablation structures 101 can fit within and be conveyed through an endoscope internal working channel 211. In each case, the ablation structure 101 and an accompanying deflection mechanism can be conveyed through the internal working channel 211 in a dimensionally compacted first configuration that is capable of expansion to a second radially expanded configuration upon exiting the distal end 110 of the endoscope 111 (For example, see FIGS. 34A, 34B, 35A, 35B, 36A, and 36B).

As shown in FIG. 34B, in one embodiment, the deflection mechanism is an inflation member 105, to which the ablation structure 101 can be integrated within or mounted/attached to, for example by etching, mounting or bonding. The inflation member 105 can be, for example, a compliant, non-compliant or semi-compliant balloon.

As shown in FIGS. 35B and 35B, in another embodiment, the deflection mechanism is an expandable member 209 that can expand to a second desired arrangement and configuration. As shown in FIG. 35B, the expandable member 209, can be an expandable stent, frame or cage device, to which an ablation structure 101 is mounted or integrated. For example, where the expandable member 209 is a wire cage, the wires can be a component of a bipolar circuit to provide the ablation structure 101 feature. Alternatively, the cage can have a flexible electrode circuit bonded or can be attached to an outer or inner surface of the cage to provide an ablation structure 101 that is an electrode. As shown in FIG. 36B, the expandable member 209, can be a folded or rolled series of hoops including or having an attached ablation structure 101 that expands upon exiting the endoscope distal end 110.

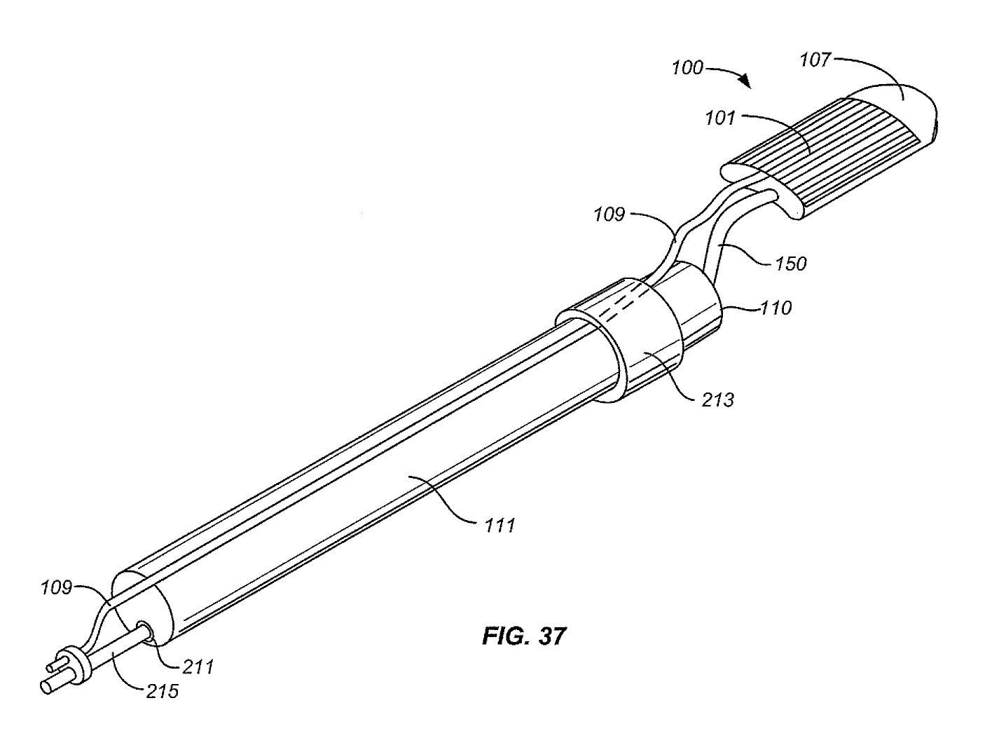

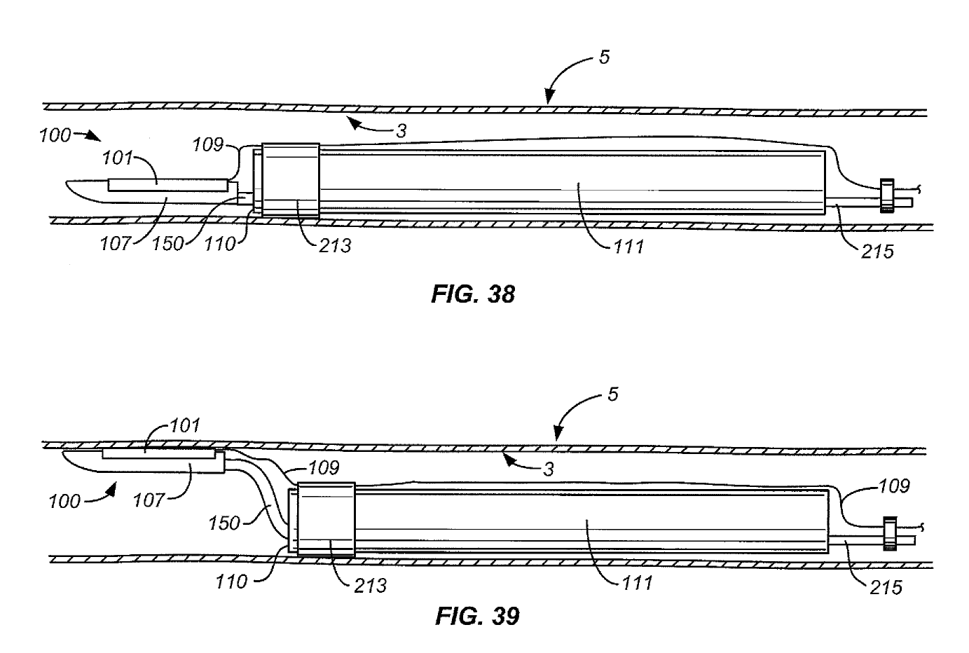

As further illustrated in FIGS. 37-39, the ablation structure 101 can be supported with a channel of the endoscope 111. In one embodiment as shown in FIGS. 37-39, an ablation device 100 includes a deflection member 150 that supports an attached housing 107 and ablation structure 101. As shown in FIG. 39, the endoscope 111 includes an internal working channel 211 suitable for advancing or retreating the deflection member 150 which is connected to an internal coupling mechanism 215 of the ablation device 100. FIGS. 37 and 39 both show a deflection member 150 including a bent region of the deflection member 150 in a deployed position, wherein the deflection member 150 bent region is positioned external to the endoscope distal end 110. FIG. 38 shows the deflection member 150 in an undeployed position, wherein the deflection member 150 bent region is positioned internal to the endoscope 111. The ablation structure 101 is thus supported with a channel of the endoscope 111 (the internal working channel 211 of the endoscope 111) by way of the deflection member 150 and the connected internal coupling mechanism 215 of the ablation device 100.

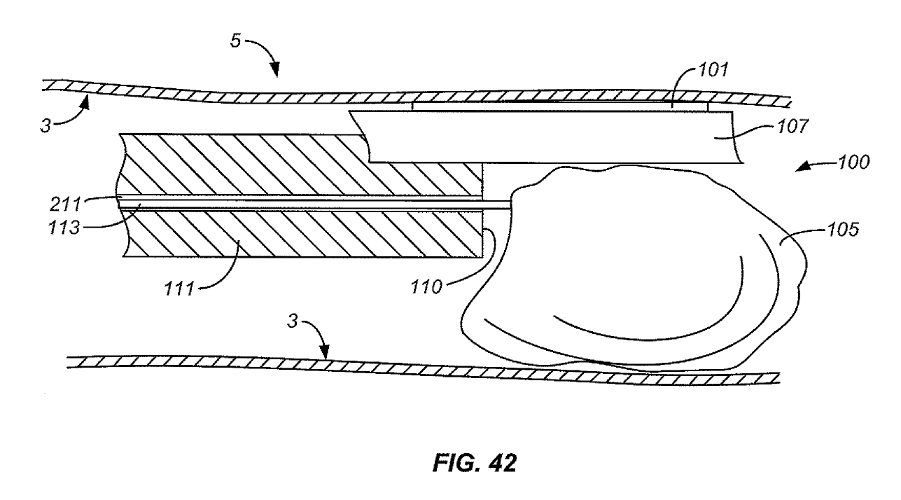

In addition, when the deflection member 150 is advanced or moved proximally or distally within the endoscope internal working channel 211, the deflection member 150 is accordingly advanced through a channel of the endoscope 111. In another implementation, as shown in FIG. 42, wherein the deflection mechanism is an inflatable member 105 (shown in a deployed configuration) coupled to an inflation line 113, the inflation line 113 can be disposed within the endoscope internal working channel 211. In yet another implementation, both the inflatable member 105 (in an undeployed configuration) and inflation line 113 can be advanced within the internal working channel 211 either proximally or distally in relation to the endoscope 111. Conductive wires 109 can pass through the working channel (not shown) or outside as shown in FIG. 37.

As shown in FIG. 41, in another implementation the endoscope 111 includes an internal working channel 211 suitable for supporting the ablation housing 107 and ablation structure 101 which are connected to an internal coupling mechanism 215 of the ablation device 100. As such, the connected ablation structure 101 is supported within a channel of the endoscope 111. Additionally as shown in FIG. 41, the housing 107 and ablation structure 101 can further be supported by an external region of the endoscope 111, wherein the internal coupling mechanism 215 is adapted and configured to position the housing 107 in contact with the external region of the endoscope 111. The internal coupling mechanism 215 can be cannulated (not shown) to facilitate use of the working channel to aspirate and flow in fluids or air.

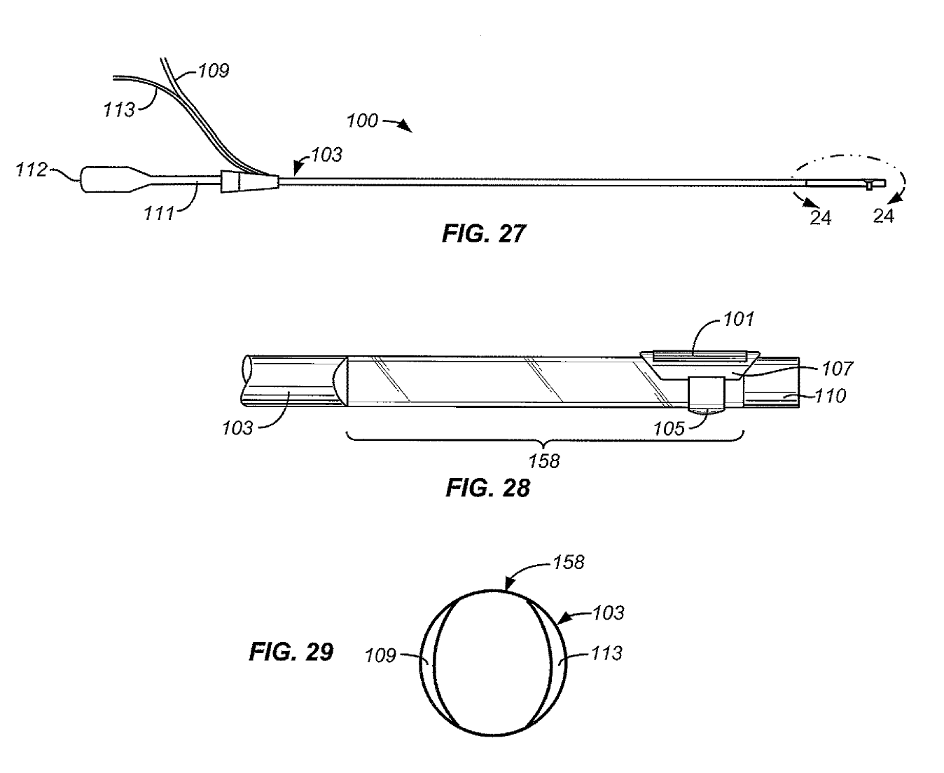

In another ablation method, an additional step includes moving the ablation structure 101 with respect to the endoscope 111 within a lesion region. As illustrated in FIGS. 27, 28, 30, 32, and 47, and as discussed below, a sheath 103 of the ablation device 100 to which the ablation structure 101 is attached can enable moving the ablation structure 101 with respect to the endoscope 111. Further, as illustrated in FIGS. 34A, 35A, 36A, 37, 38, 39, and 41, and discussed above, an internal working channel 211 of the endoscope 111 through which at least a part of the ablation device 100 is disposed can enable moving the ablations structure 101 with respect to the endoscope 111.

Referring to FIGS. 11, 31, 42, and 44, in yet another method, the step of deflecting the ablation structure 101 toward a tissue surface 3 includes inflating an inflation member 105 of the ablation device 100 within a lesion region of a gastrointestinal tract. The inflation member 105 can be arranged and configured to be reversibly inflatable. The inflation member 105 can be inserted along with the ablation structure 101 into an alimentary tract in a collapsed configuration and expanded upon localization at a pre-selected treatment area. In one implementation, the inflation member 105 is a balloon. For example, in FIGS. 11, 31, 42, and 44 it is shown how deflecting the ablation structure 101 toward a tissue surface 3 is achieved when the inflation member 105 is inflated or deployed. As illustrated in FIGS. 11, 31, 42, and 44, upon sufficient inflation, the inflation member 105 contacts a tissue surface 3 consequently deflecting the ablation structure 101 which contacts an opposing tissue surface 3.

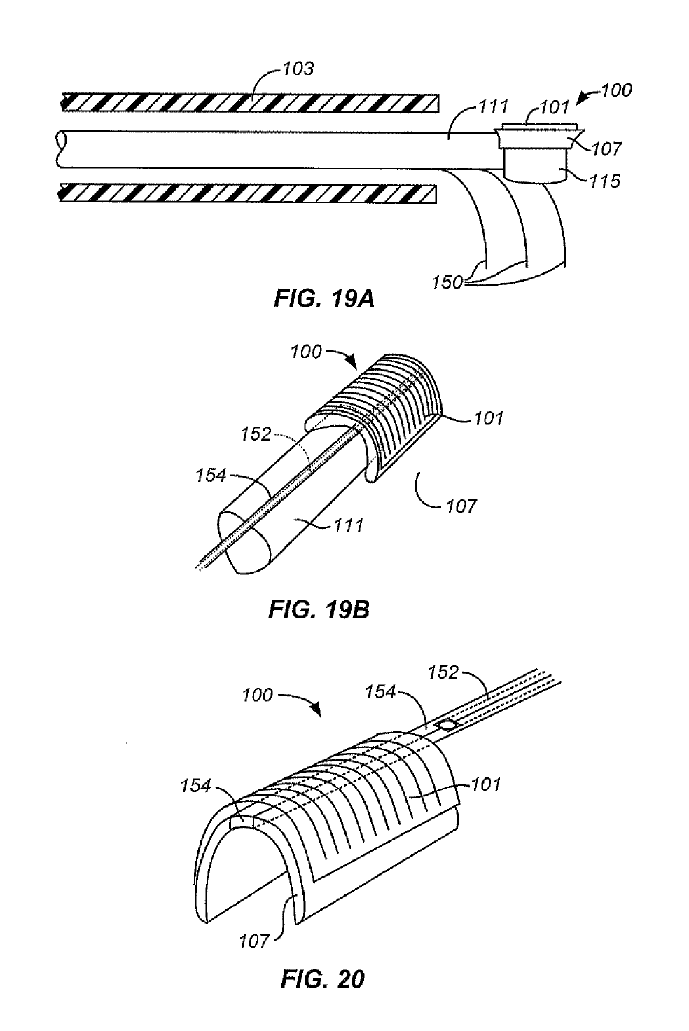

As shown in FIGS. 19B, 20, 35, 36 and discussed above, in a further method, the step of deflecting the ablation structure 101 includes expanding a deflection structure or deflection member 150. In one implementation, as shown in FIG. 19A the ablation device 100 includes a sheath 103, wherein the sheath 103 is arranged and configured to receive the deflection member 150, the endoscope 111 and ablation structure 101 internally to the sheath 103. In one implementation, the deflection member 150 is a shape memory alloy, for example, Nitinol. The flexible extensions of the deflection member 150 in this embodiment can be coupled to the endoscope, an elastomeric sheath 115 of the ablation device 100 (shown in FIG. 19A) or any part of the device 100, including the ablation housing 107.

As shown in FIGS. 34, 35, 36, 37, 38, and 39, and discussed above, in a further method, the step of deflecting the ablation structure 101 includes moving a deflection structure or deflection member 150.

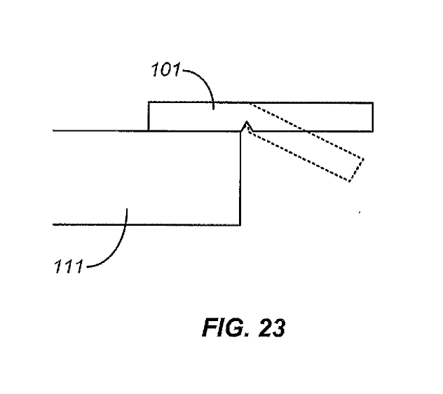

Briefly, in each case moving the deflection 150 is used to change the deflection member 150 from a non-deployed to a deployed configuration. As shown in FIG. 23, in one embodiment, deflecting the ablation structure 101 includes a flexing point in the ablation structure 101, wherein the ablation structure 101 can deflect in response to, for example, resistance met in contacting a tissue surface 3.

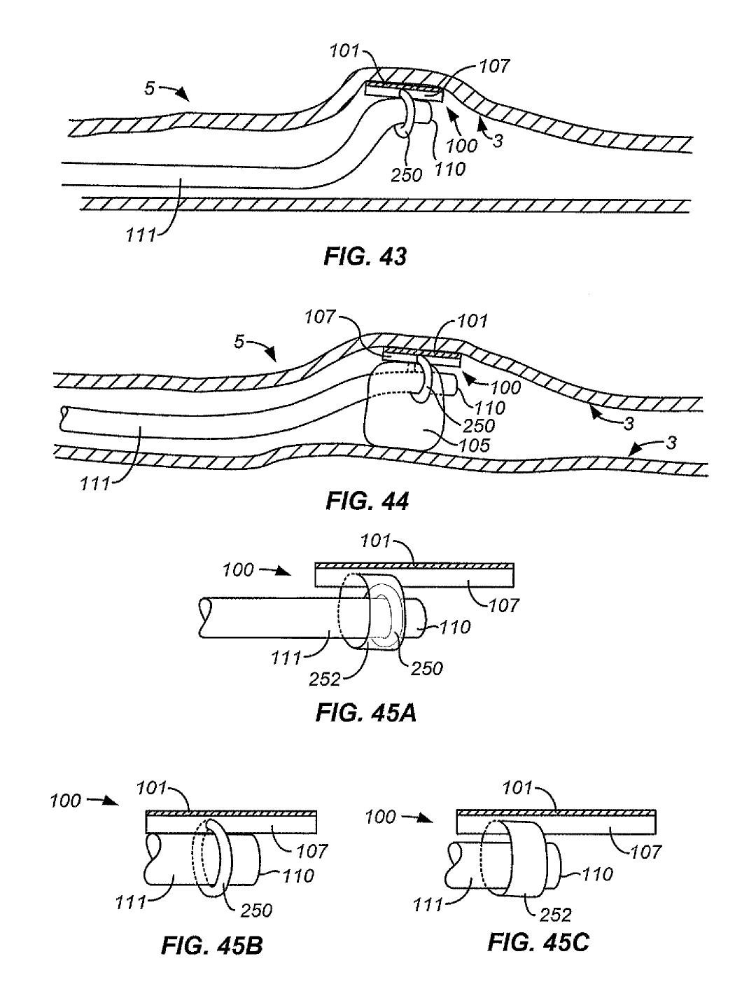

As shown in FIGS. 43, 44, and 45A-45C and as discussed in further detail below, in another method, the step of deflecting the ablation structure 101 includes rotating, pivoting, turning or spinning the ablation structure 101 with respect to the endoscope 111 along their respective and parallel longitudinal axes. Deflection of the ablation structure 101 with respect to the endoscope 111 can occur in combination with the endoscope 111 distal end 110 deflecting with respect to a target lesion site. Also, the ablation structure 101 can deflect in combination with an inflation member 105 used to achieve apposition of the ablation device 100 to the tissue. In some embodiments, the step of deflecting the ablation structure 101 may additionally include any combination of the above disclosed deflecting steps.

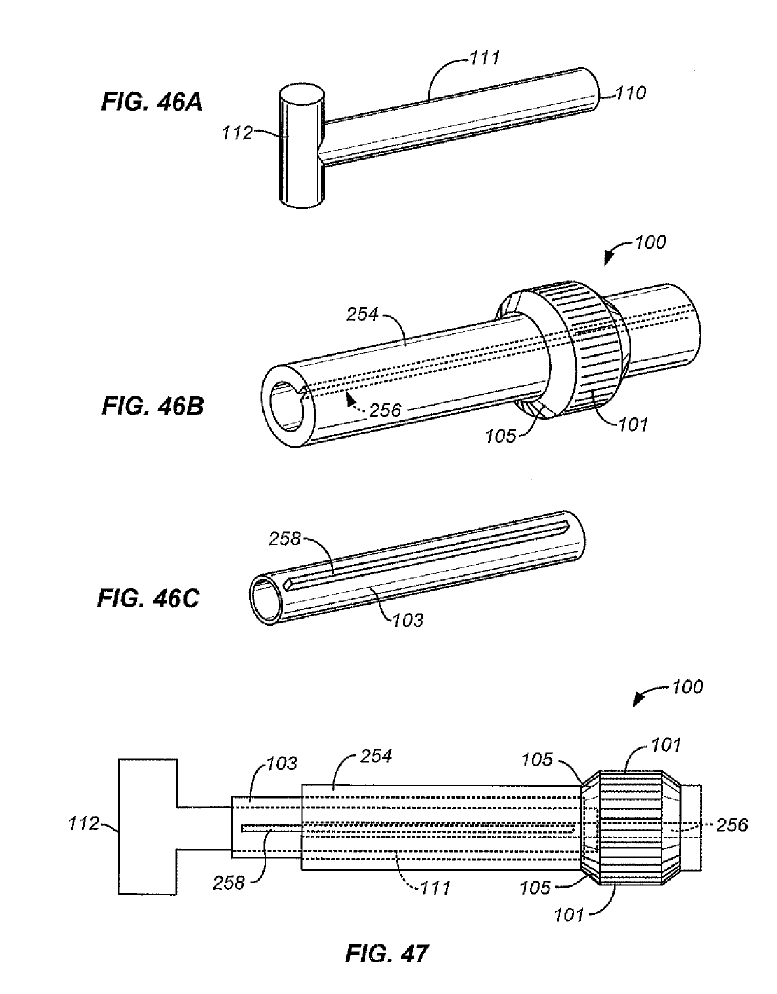

As shown in FIGS. 19, 20, 21, 22, 34A, 34B, 35A, 35B, 36A, 36B, 46B, and 47, in another ablation method, an additional step includes moving the ablation structure 101 from a first configuration to a second radially expanded configuration. The details regarding radial expansion of the ablation structure 101 shown in FIGS. 19, 20, 21, and 22 are described below, while the details for FIGS. 34A, 34B, 35A, 35B, 36A, and 36B are described above. Additionally, as shown in FIGS. 46B and 47 the ablation structure 101 can be arranged in a first configuration wherein the ablation structure 101 is coupled directly or alternatively through an housing 107 (not shown) to an inflation member 105 attached to a catheter 254. In an undeployed configuration as shown in FIGS. 46B and 47, the non-inflated inflation member 105 and ablation structure 101 have a relatively low profile in relation to the endoscope 111. When deployed, the inflation member 105 moves the ablation structure 101 to a second radially expanded configuration (not shown).

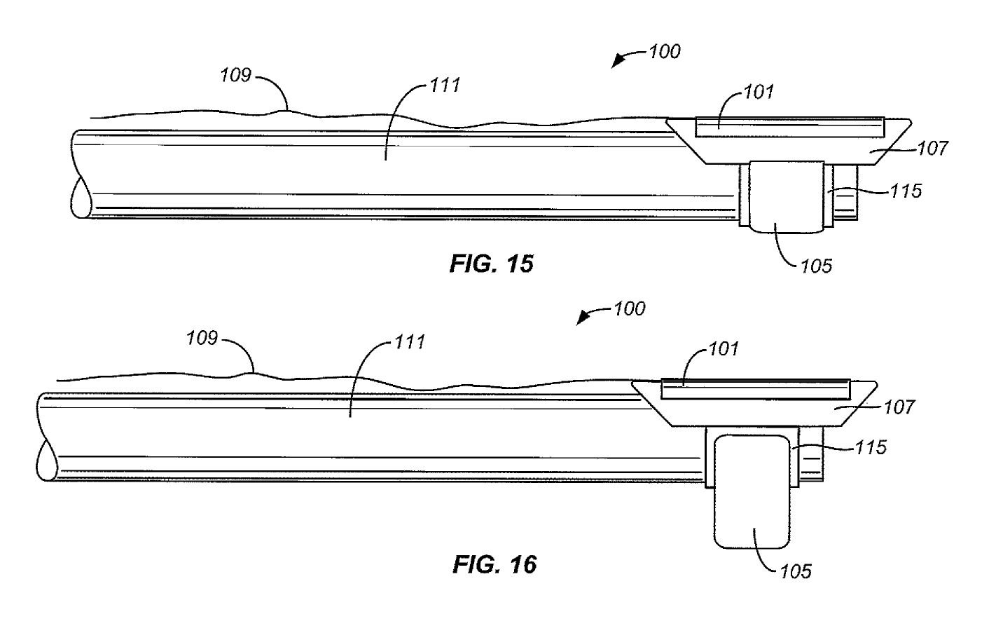

As shown in FIGS. 15, 16, 40, 43, 44, 45A-45C, 46B, and 47, in a further method, an additional step includes attaching the ablation structure 101 to the endoscope 111. As shown in FIGS. 15 and 16, attachment of the ablation structure 101 to the endoscope 111 can also be by way of an elastomeric sheath 115 The elastomeric sheath 115 can removably hold the ablation structure 101 in a desired position on the endoscope 111. The elastomeric sheath 115 can be arranged and configured to fit over the endoscope distal end 110. As shown in FIGS. 15 and 16, the inflation member 105 can be attached to the elastomeric sheath 115 or alternatively the inflation member 105 can also act as the “elastomeric sheath” (not shown).

In another method, the step of attaching the ablation structure 101 to the endoscope 111 includes attaching the ablation structure 101 to an outside surface of the endoscope. Alternatively, the attaching step can include, for example, attaching to an inside surface, an outside or inside feature of the endoscope, or any combinations of the above. Lubricants such as water, IPA, jelly, or oil may be use to aid attachment and removal of the ablation device from the endoscope.

As shown in FIG. 41, in a further method, the step of attaching the ablation structure 101 to the endoscope 111, includes an ablation structure 101 having an attached rolled sheath 116, wherein attaching the ablation structure 101 to the endoscope 111 includes unrolling the sheath 116 over an outside surface of the endoscope 111. The rolled sheath 116 can additionally cover the electrical connections 109 of the ablation device 100 along a length of the endoscope 111 (see FIG. 41). In a related method, the ablation structure 101 is attached to the endoscope 111 by an attaching step including unrolling the rolled sheath 116 over an outside surface of the endoscope 111 and part of the ablation structure 101. This structure may also be used to mount the ablation structure on one or more fingers of a user (as shown in FIG. 60).

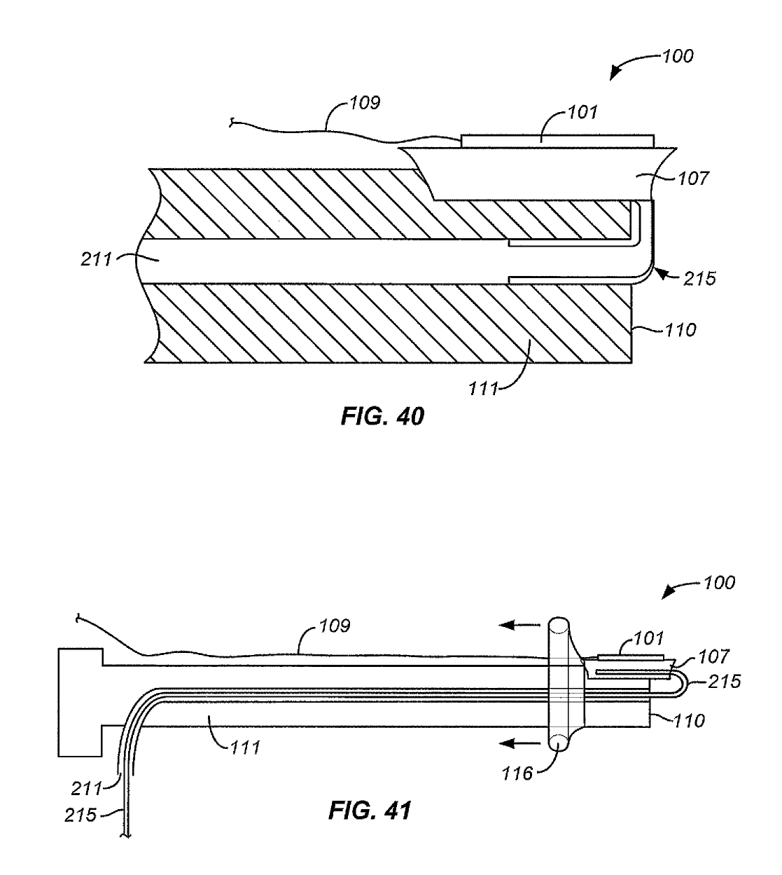

In another method, as shown in FIG. 40, the step of attaching the ablation structure 101 to the endoscope 111 includes attaching the ablation structure 101 to a channel of the endoscope. As shown in FIG. 40, in one implementation, the housing 107 and ablation structure 101 are coupled to an internal coupling mechanism 215 that can be positioned within an internal working channel 211 of the endoscope 111. The internal coupling mechanism 215 in FIG. 40 is shown as attached to the internal working channel 211 at the endoscope distal end 110. In this embodiment, the housing 107 and ablation structure 101 are shown in alignment with and coupled to an outside surface of the endoscope 111 near the distal end 110.

In one method of ablating tissue in the alimentary tract, the tissue surface 3 can include a first treatment area and activation of the ablation structure 101 step can include activation of the ablation structure 101 to ablate the first treatment area, and further include moving the ablation structure 101 to a second area without removing the ablation structure 101 from the patient and activating the ablation structure 101 to ablate the second tissue area 3. Moving, in this sense, refers to moving the ablational structure to the locale of a target site, and thereafter, further moving of the structure into a therapeutically effected position can be performed variously by inflating a balloon member, or deflection or inflating a deflection member, as described in detail elsewhere. For example, two or more areas of the tissue surface 3 of a target area can be ablated by directing the ablation structure 101 to the first target region and then activating the ablation structure 101 to ablate the tissue surface 3. Then, without removing the ablation structure 101 from the patient, the ablation structure 101 can be directed to the second target area for ablation of the appropriate region of the tissue surface 3.

In general, in another aspect, an ablation device 100 is provided that includes an ablation structure 101 removably coupled to an endoscope distal end 110, and a deflection mechanism adapted and configured to move the ablation structure 101 toward a tissue surface 3 (see for example, FIGS. 5-19, 22, 22, 27-29, 30-32, 34A, 35A, 36A, 37, 38, 39, 42, 44, and 47).

In a related embodiment, the ablation device 100 additionally includes an ablation structure movement mechanism adapted to move the ablation structure 101 with respect to the endoscope 111. As discussed below and shown in FIGS. 26-28, and 30-32, the ablation structure movement mechanism can be a sheath 103 to which the ablation structure 101 is attached, wherein the sheath 103 is arranged and configured to move the ablation structure 101 with respect to an endoscope 111 received within the sheath 103. Alternatively, as discussed above and shown in FIGS. 34A, 35A, 36A, and 37-39, the ablation structure movement mechanism can be in the form of an internal coupling mechanism 215 of the ablation structure 100, wherein the ablation structure is connected to the internal coupling mechanism 215 and at least a portion of the internal coupling mechanism 215 is disposed internally to the endoscope.

In another embodiment, the ablation device 100 additionally includes a coupling mechanism designed to fit over an outside surface of an endoscope 111, to couple the ablation structure 101 with the endoscope 111. As discussed above, a spiral sheath 104, an elastomeric sheath 115, a rolled sheath 116 and an internal coupling mechanism as shown in FIGS. 15, 16, 40, and 41 respectively, are examples of such coupling mechanisms. In a particular embodiment, the coupling mechanism includes a sheath 103 capable of supporting the ablation structure 101. The sheath 103 can be tubing, a catheter or other suitable elongate members. The sheath 103 can be arranged and configured so that it can be moved independently of an associated endoscope.

As shown in FIG. 41, in another embodiment, the sheath 103 can be arranged and configured as a rolled sheath 116 that can be unrolled over the outside surface of the endoscope. In use, a rolled sheath 116 connected to the ablation device 100, for example at substantially near the proximal end of the housing 107 (from the perspective of an operator of the device), can be unrolled from such a position and continue to be unrolled toward the proximal end 112 of the endoscope 111 (see FIG. 47). In this way, the rolled sheath 116 can be caused to contact and cover all or a portion of the length of the endoscope 111 (not shown). Additionally, as the rolled sheath 116 is unrolled along the endoscope 111, it can sandwich the electrical connections 109 between the rolled sheath 116 and the endoscope 111 (see generally FIG. 41).

In another embodiment, as shown in FIGS. 30 and 31, the sheath 103 can be arranged and configured to support a deflection mechanism wherein the deflection mechanism includes a deflection structure or deflection member 150. As illustrated in FIGS. 30 and 31, where the deflection member 150 is an inflation member 105, the inflation member 105 can be directly attached to the sheath 103. As shown in each case, the inflation member 105 is positioned opposite the placement of the ablation structure 101, which is also attached to the sheath 103. This configuration of the sheath 103 provides support for the inflation member 105 and the ablation structure 101 irrespective of the positioning of the endoscope distal end 110. For example, as shown in FIG. 30, the endoscope distal end 110 can be positioned to provide a gap between the distal end 110 and a distal end of the sheath 103 where the ablation structure 101 and inflation member 105 are positioned. In contrast, as shown in FIG. 31 the endoscope distal end 110 can extend through and beyond the distal end of the sheath 103.

In another embodiment, as shown in FIG. 26, the sheath 103 can be elongated. FIG. 26 illustrates a sheath including electrical connections 109 and an inflation line 113. The sheath 103 may include pneumatic and/or over extruded wires impregnated within the sheath 103. In use, the sheath 103 can be introduced first into an alimentary tract, wherein the sheath 103 serves as a catheter like guide for introduction of the endoscope 111 within the sheath 103. Alternatively, the endoscope 111 may be introduced first and thereby serve as a guidewire for the sheath 103 to be introduced over. FIG. 26 also shows attachment of an inflation member 105 to the sheath 103, in an arrangement wherein the ablation structure 101 is attached to the inflation member 105 opposite the sheath 103 attachment point.

In embodiments shown in FIGS. 27 and 28, the sheath 103 includes an optically transmissive portion 158 adapted and configured to cooperate with a visual channel of an endoscope 111. For example, the sheath 103 may be made of clear, translucent or transparent polymeric tubing including PVC, acrylic, and Pebax® (a polyether block amide). As shown in FIG. 24, one component of an endoscope 111 can be a visual channel 161 that provides visual imaging of a tissue surface 3 as imaged from the endoscope distal end 110. For example, the transmissive portion 158 can allow visualization of the wall of an esophagus 3 through the transmissive portion 158 of the sheath 103. As shown in FIG. 28 and in the cross-section view provided in FIG. 29, the sheaths 103 shown in FIGS. 27 and 28, include an optically transmissive portion 158 arranged and configured to provide viewing of tissue surfaces 3 through the wall of the sheath 103, with the aid of an internally disposed endoscope 111 having a visual channel 161. Also shown in cross-section in FIG. 29 are portions of the sheath 103 through which electrical connections 109 and an inflation line 113 can pass. These features may be imbedded into the sheath 103 inner-wall or attached to the sheath 103 inner wall. As shown in FIG. 27, the sheath 103 including a transmissive portion 158 can extend past the endoscope distal tip 110. Alternatively, as shown in FIGS. 27, 28, and 31, the endoscope distal end 110 can extend distally past the transmissive portion 158 of the sheath 103.

In another implementation, the transmissive portion 158 of the sheath 103 can be reinforced structurally with coil or braid elements incorporated therein to prevent ovalization and/or collapsing of the sheath 103, particularly while deflecting the ablation device 100.

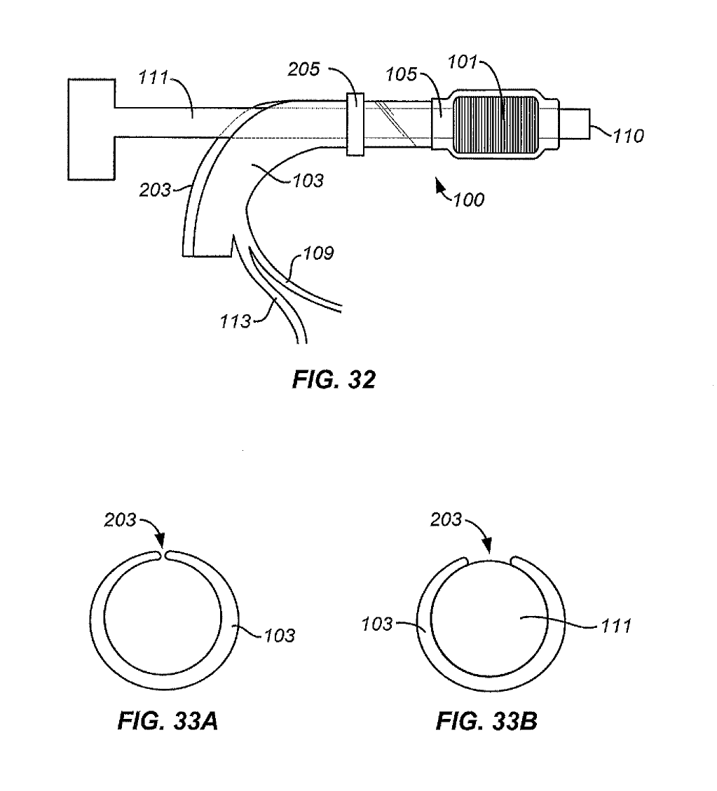

In a further embodiment, the sheath 103 includes a slit 203 formed in a proximal portion of the sheath 103, the slit 203 being designed to open to admit an endoscope distal end 110 into the sheath 103. As shown in FIG. 32 the proximal portion of the sheath 103 can include a perforation region or slit 203. The slit 203 can extend partially of fully along the length of the sheath 103. The slit 203 enables the sheath 103 to be pulled back, or opened when, for example introducing an endoscope 111 into the sheath 103. In one implementation, as shown in FIG. 32, the sheath 103 additionally includes a locking collar 205 for locking the sheath 103 in a desired position in respect to the endoscope 111.

As shown in FIGS. 33A and 33B, the distal portion of the sheath 103 can have a smaller outer diameter than a, proximal portion of the sheath 103, the distal portion of the sheath 103 being adapted and configured to be expanded when an endoscope 111 is inserted into it (not shown). This embodiment can aid in accessing an endoscope 111 in a case where the sheath 103 is advanced first into a target site. Since the distal end of the sheath 103 is smaller in diameter, but includes a slit 203, the sheath 103 can accept a larger outside diameter endoscope 111 because when the endoscope 111 is advanced, the slit 203 of the sheath 103 allows for widening of the sheath 103.

In general, in another aspect, a method of ablating tissue in within the alimentary tract includes advancing an ablation structure 101 into the alimentary tract while supporting the ablation structure 101 with an endoscope 111. The endoscope distal end 110 can be bent to move the ablation structure 101 into contact with a tissue surface followed by activation of the ablation structure 101 to ablate the tissue surface 3 (see e.g., FIG. 43). In a particular embodiment, the ablation structure 101 includes a plurality of electrodes and the activating step includes applying energy to the electrodes.

In general, in another aspect the coupling mechanism is designed to fit over an outside surface of an endoscope 111, to couple the ablation structure 101 with the endoscope 111, rather than being for example, a sheath (as discussed above), and is adapted and configured to provide a certain freedom of movement to the ablation structure 101, including but not limited to flexing and/or rotating and/or pivoting with respect to the endoscope 111 when coupled to the endoscope 111. The freedom of movement is with respect to one, two, or three axes, thereby providing one, two, or three degrees of freedom. Non-limiting examples of suitable coupling mechanisms include a flex joint, pin joint, U-joint, ball joint, or any combination thereof. The following described coupling mechanism embodiments advantageously provide for a substantially uniform apposition force between a supporting endoscope 111 and an ablation structure 101 when localized at a target tissue surface 3.

As shown in FIGS. 43, 44, 45A, and 45B, the coupling mechanism can be a ring 250 attached to the housing 107 and the endoscope 111, wherein the housing 107 is adapted and configured to flex, rotate or pivot about the ring 250. Alternatively, the coupling may be configured for hard held or finger mounted use. For example, as illustrated in FIG. 43, where the ablation device 100 is coupled to a deflectable distal end 110 of an endoscope 111 by a ring 250, when the device 100 is deflected toward the target tissue surface 3, the housing 107 upon contact aligns the ablation structure 101 with the tissue surface 3 by flexing, rotating or pivoting about the ring 250 coupling. In these embodiments, the endoscope and the housing that supports the ablation structure both have their own longitudinal axis, and these axes are situated parallel to each other. The coupling mechanism that attaches the housing to the endoscope allows a pivoting movement between the longitudinal axis of the housing and the longitudinal axis of the endoscope. Advantageously, sufficient contact pressure provided by deflection of the distal end 110 of the endoscope 101 can produce a desired degree of contact between the ablation structure 101 and the tissue surface 3, irrespective of the precise alignment of the distal end 112 in respect to a plane of the tissue surface 3 to be treated. For the purposes of this disclosure, a “desired degree of contact”, “desired contact”, “therapeutic contact”, or “therapeutically effective contact” between the ablation structure 101 and the tissue surface 3, includes complete or substantially-complete contact between all or a portion of a predetermined target on the tissue surface 3 (e.g. a lesion or lesions in the target site) by all or a portion of the ablation structure 101.

As shown in FIG. 44, in a different yet related embodiment, where the deflection mechanism of the ablation device 100 is an inflatable member 105, a ring 250 coupling allows for flexing, rotating or pivoting of the housing 107 and ablation structure 101. As in the previous case, sufficient contact pressure provided through deflection, here by the inflatable member 105, can produce a desired degree of contact between the ablation structure 101 and the tissue surface 3. Again, advantageously, the desired contact can be achieved irrespective of the precise alignment of the deflected endoscope 111 distal end 110 in respect to a plane of the tissue surface 3 to be treated, because of the flexing, rotating or pivoting provided by the ring 250 coupling.

As shown in FIG. 45A, in a related embodiment, the coupling mechanism between the ablation device 100 and an endoscope 111 can be an elastic band 252, wherein the housing 107 of the device 100 is flexibly coupled to the elastic band 252. For example, as illustrated in FIG. 45C, where the ablation device 100 is coupled to a distal end 110 of an endoscope 111 by an elastic band 252, when the device 100 is deflected toward a tissue surface 3, alignment between the housing 107 and accordingly the ablation structure 101 and the tissue surface 3, can be achieved by flexing about the elastic band 252 coupling. Once more, advantageously, the desired contact can be achieved irrespective of the precise alignment of the deflected endoscope's 111 distal end 110 in respect to a plane of the tissue surface 3 to be treated, because of the flexing capability provided by the elastic band 252 coupling.

As shown in FIG. 45A, in another related embodiment, the coupling mechanism between the ablation device 100 and an endoscope 111 can be a combination of a ring 250 and an elastic band 252, wherein the housing 107 of the device 100 is coupled to the elastic band 252. For example, as illustrated in FIG. 45A, where the ablation device 100 is coupled to a distal end 110 of an endoscope 111 by an elastic band 252, when the device 100 is deflected toward a tissue surface 3 containing a lesion to be treated, alignment between the housing 107 and accordingly the ablation structure 101, and the tissue surface 3 by flexing, rotating or pivoting about the ring 250 and the elastic band 252 coupling can be achieved. Again, advantageously, the desired contact can be achieved irrespective of the precise alignment of the deflected endoscope 111 distal end 110 in respect to a plane of the tissue surface 3 to be treated, because of the flexing rotating or pivoting provided by the elastic band 252 coupling.

In another embodiment, the ablation device 100 additionally includes an alternative coupling mechanism between the ablation device 100 and an endoscope 111 that is arranged and configured to fit within a channel of an endoscope 111. The coupling mechanism can be an internal coupling mechanism 215 and can be configured and arranged to couple the ablation structure 101 within an internal working channel 211 of an endoscope 111 (see FIG. 37 and as discussed above).

As shown in FIGS. 34A, 34B, 35A, 35B, 36A, and 36B, in one embodiment of such a coupling mechanism, the ablation structure 101 is adapted and configured to fit within the endoscope internal working channel 211. Additionally, as shown in FIGS. 34A, 34B, 35A, 35B, 36A, and 36B, in a related embodiment, the deflection mechanism is also adapted and configured to fit within the endoscope internal working channel 211.

In each of the embodiments described above and shown in FIGS. 34A, 34B, 35A, 35B, 36A, and 36B, after expansion of the inflatable member 105 or expandable member 209 and subsequent treatment of a target tissue 3, the coupling means can further serve as a means to draw, pull or retrieve the ablation structure 101 and deflection mechanism back into the endoscope internal working channel 211. Furthermore, in addition to providing coupling of the ablation structure 101 with the endoscope internal working channel 112, the coupling mechanism can include electrical connections 109 to provide energy to the ablation structure 101.

In a related embodiment, again wherein the ablation device 100 additionally includes a coupling mechanism adapted and configured to fit within a channel of an endoscope 111, the coupling mechanism can include a shape memory member and the deflection mechanism can include a bent portion of the shape memory member. As shown in FIGS. 37-39, the coupling mechanism can be an internal coupling mechanism 215. As shown, the internal coupling mechanism 215 can be disposed within an endoscope internal working channel 211 and extend beyond the endoscope distal end 100. Additionally, the internal coupling mechanism 215 can be connected to a deflection mechanism that is a deflection member 150. The deflection member 150 can include a bent portion and can be connected to the housing 107. As shown in FIG. 38 and discussed above, the bent portion of the deflection member 150 can be disposed within the endoscope internal working channel 211, causing the ablation structure 101 to move into a non-deployed position. Upon advancing the internal coupling mechanism 215 toward the endoscope distal end 110, the shape memory nature of the deflection member 150 facilitates deployment of the ablation structure 101 to a position suitable for ablation.

In general, in one aspect, the ablation structure 101 of the ablation device 100 includes an optically transmissive portion 158 adapted and configured to cooperate with a visual channel of an endoscope 111. As shown in FIGS. 27-31 and discussed above, the optically transmissive portion 158 can be a sheath 103 of the ablation device 100.

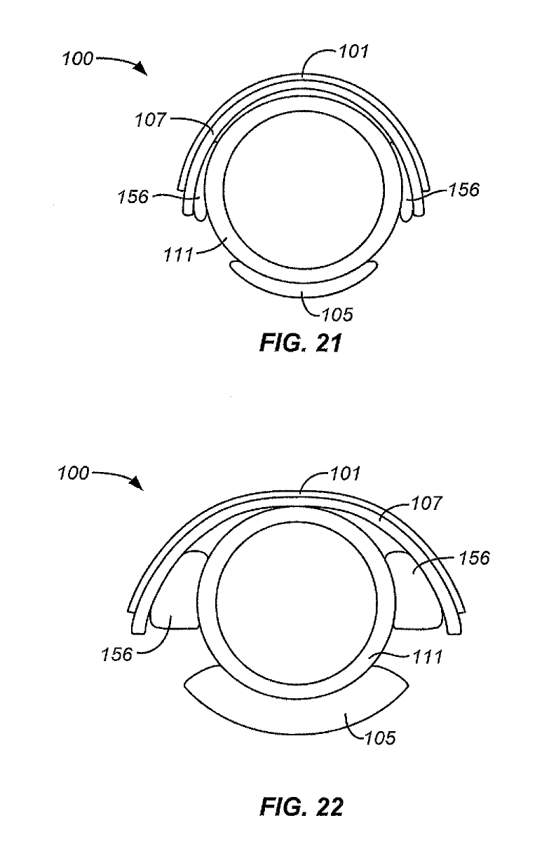

In one embodiment, the ablation structure 101 of the ablation device 100 is further adapted and configured to move from a first configuration to a second radially expanded configuration. As shown in FIGS. 19-22, the ablation structure 101 and housing 107 can be designed to reversibly move from a first less radially expanded configuration (see FIGS. 20 and 21) to a second radially expanded configuration useful for ablation. Foldable or deflectable configurations that provide for reversible radial expansion of the housing 107 and the ablation structure 101 can facilitate access to tissue surfaces because of reduced size. Additionally, foldable or deflectable configurations are helpful in regard to cleaning, introduction, retrieval, and repositioning of the device in the lesion containing regions.

The ablation device 100 shown in FIGS. 19B and 20 includes an ablation structure actuator 152 arranged and configured to move the ablation structure 101 from the first configuration (see FIG. 20) to a second radially-expanded configuration (see FIG. 21). As shown (FIGS. 19B and 20), the actuator 152 can be elongate and designed to work with a receiver 154 arranged and configured to receive the actuator 152. The actuator 152 can be a wire, rod or other suitable elongate structure. Alternatively, the actuator 152 can be a hydraulic actuation means with or without a balloon component. In a particular embodiment, the actuator 152 is a stiffening wire.

As illustrated in FIG. 20, before the actuator 152 is disposed within the portion of receiver 154 attached to the housing 107, both the housing 107 and the ablation structure 101 are in a first position having a first configuration. As illustrated in FIG. 21, after the actuator 152 is partially or fully introduced into the receiver 154, the housing 107 and the ablation structure 101 are consequently changed to a second radially expanded configuration relative to the first configuration. Introduction of the actuator 152 into the receiver 154 can force the portions of the housing 107 and ablation structure 101 flanking the receiver 154 to expand radially (see FIG. 19). In one embodiment, the housing 107 is heat set in a flexed first configuration suitable for positioning the ablation device 100 near a target tissue surface 3. After a target tissue surface 3 has been reached, the actuator 152 can be introduced into the receiver 154 to achieve the second radially expanded configuration which is useful for ablation of the tissue surface 3.

In a related alternative embodiment, the housing 107 and ablation structure 101 include an unconstrained shape that is radially expanded and includes one or more flex points to allow for collapsed or reduced radial expansion when positioned distally to the distal end 110 of an endoscope 111 and compressed by an elastomeric sheath 115 (not shown).

As shown in FIGS. 21 and 22, in another embodiment, the ablation structure 101 of the ablation device 100 is adapted and configured to move from a first configuration to a second radially expanded configuration wherein the ablation device 100 further includes an expandable member 156. The expandable member 156 can be positioned between the housing 107 and the endoscope 111, where in unexpanded form, the ablation structure 101 is accordingly configured in a first configuration. Upon expansion of the expandable member 156, the ablation structure 101 configuration is changed to a second radially expanded configuration (see FIG. 21).

In one embodiment, the deflection mechanism of the ablation device 100 includes an inflatable inflation member 105. As shown in FIGS. 11, 21, 22, 25B, 27, 28, 30, 31, 34A, 34B, 42, 44, 46, and 47 and discussed above, the inflation member 105 can facilitate deflection of the device 100 in relation to a tissue surface 3.

In another embodiment, the deflection mechanism includes an expandable member 156 (see FIGS. 35B and 36B, discussed in detail above). As shown in FIG. 35B, the expandable member 209, can be an expandable stent, frame or cage device. As shown in FIG. 36B, the expandable member 209, can be an expanded series of connected hoops that can be folded or rolled prior to expansion.

In another advantageous embodiment, the ablation device 100 further comprises a torque transmission member adapted and configured to transmit torque from a proximal end of the endoscope 111 to the ablation structure 101 to rotate the ablation structure 101 about a central axis of the endoscope 111. In a particular embodiment, the torque transmission member includes first and second interlocking members adapted to resist relative movement between the endoscope 111 and the ablation structure 101 about the central axis. As shown in FIGS. 46B, 46C, and 47, in one embodiment the first interlocking member is a key 258 and the second interlocking member is a keyway 256. In one embodiment, the first interlocking member is attached to a sheath 103 surrounding the endoscope 111 and the second interlocking member is attached to a catheter 254 supporting the ablation structure 101. For example, as shown in FIGS. 46B, 46C, and 47, the key 258 can be attached to a sheath 103 surrounding the endoscope 111 and the keyway 256 can be attached to a catheter 254 supporting the ablation structure 101. In a further related embodiment, the catheter 254 and sheath 103 are arranged and configured for relative movement along the central axis of the endoscope 111. The sheath 103 can be, for example, an elastomeric sheath wherein the key 258 is attached to the outside of the sheath 103 substantially along a longitudinal axis of the sheath 103 (see FIG. 46C). In use, this embodiment provides for a 1-to-1 torque transmission of the ablation device 100 endoscope assembly 111 when the endoscope proximal end 112 is manipulated, while also providing for positioning of the ablation structure 101 either proximal or distal to the endoscope distal end 110 in situ. Additionally, the sheath 103 can be pre-loaded into the catheter 254 or loaded separately.

In general, in one aspect, an ablation device 100 is provided including an ablation structure 101, and a coupling mechanism adapted to removably couple the ablation structure 101 to a distal end 110 of an endoscope 111 and to permit the ablation structure 101 to rotate and/or pivot with respect to the endoscope when coupled to the endoscope. Various related embodiments wherein, for example, the coupling mechanism comprises a ring 250 and the ablation structure 101 is adapted to rotate and/or pivot about the ring 250; wherein the coupling mechanism comprises an elastic band 252 adapted to flex to permit the ablation structure 101 to rotate and/or pivot; wherein the ablation device 100 further includes a deflection mechanism adapted and configured to move the ablation structure 101 toward a tissue surface 3; and, wherein such a deflection mechanism includes an inflatable member, have been set out in detail above.

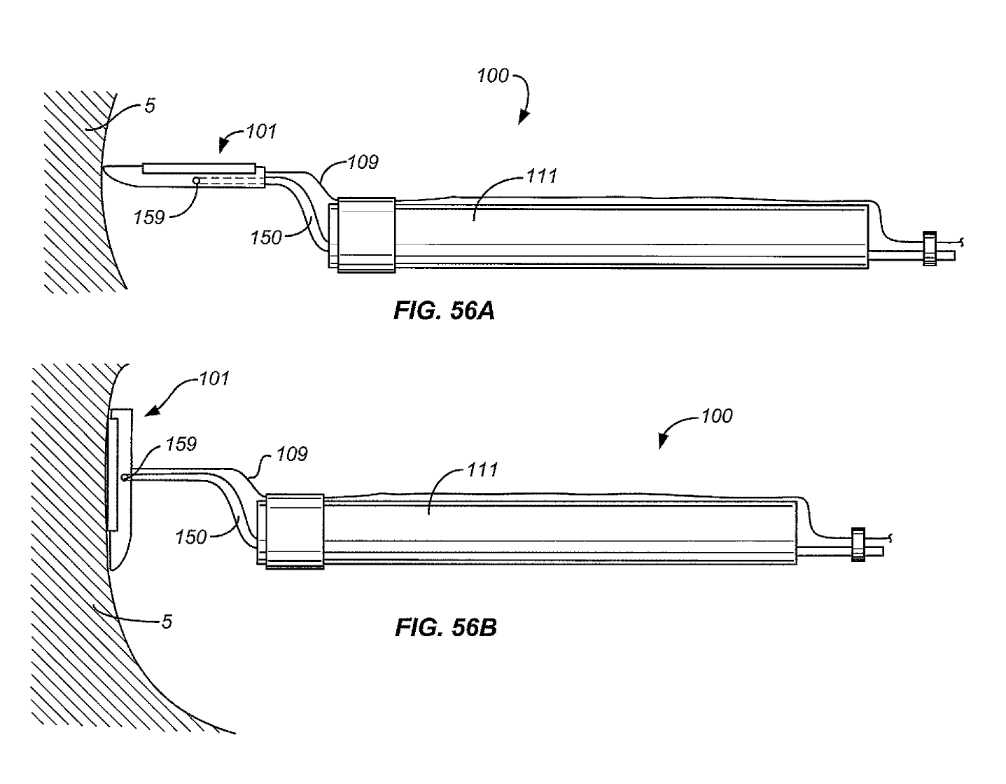

FIGS. 56A and 56B provide views of an ablational device with an ablational surface on a hinge 159 which acts in a manner similar to mechanism depicted in FIG. 43, and which allows a free pivoting movement of the ablational surface between its longitudinal axis and the longitudinal axis of an endoscope. FIG. 56A shows the device with the ablational surface 101 oriented in parallel with the endoscope, the surface having made contact with the inner surface of a gastrointestinal luminal wall 5 at a desired target area. The ablation surface 101 is supported by a deflection member 150 that can be expressed from a working channel, and withdrawn back into a working channel within the endoscope. FIG. 56B shows the device with the longitudinal axis of the ablational surface 101 oriented at about a right angle with respect to the longitudinal axis of the endoscope. This pivoting as a passive response of the ablational surface 101, as it easily rotates on hinge 159 through a flexion range of 0 degrees (parallel to the endoscope 111) to about 170 degrees. As shown, the angle of the surface is about 90 degrees with respect to the endoscope.

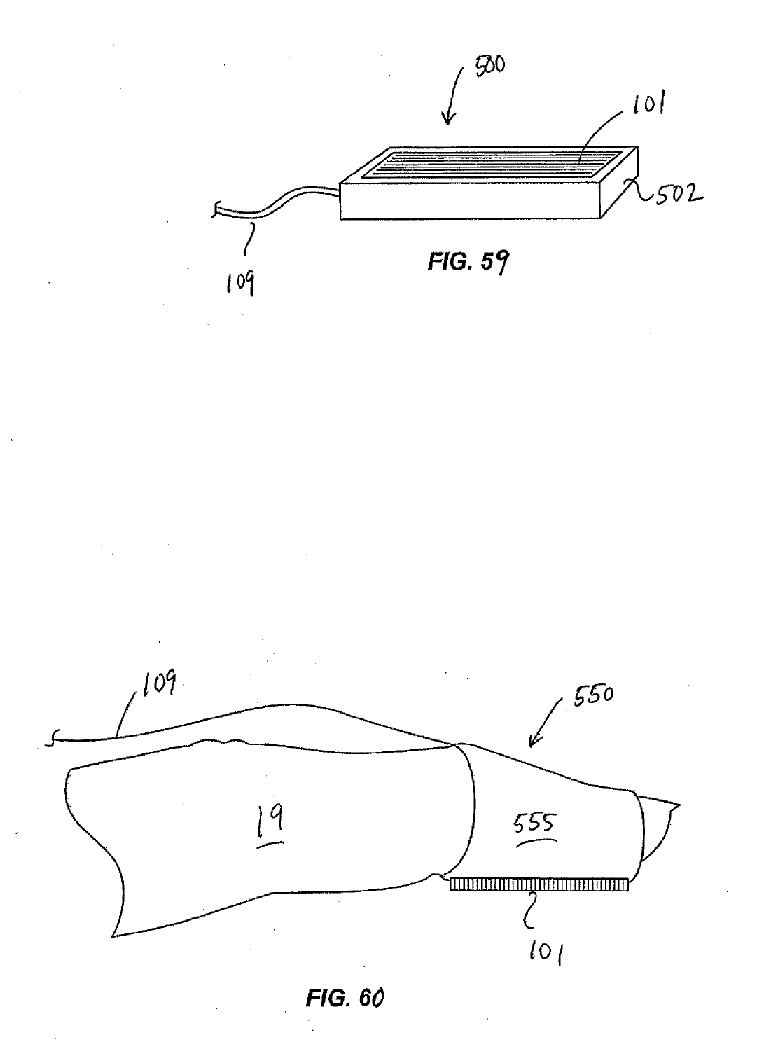

FIG. 59 is a perspective view of a hand held ablation therapy device 500. The hand held device 500 includes an electrode array 101 mounted on a substrate 502. Leads 109 attach the electrode array 101 to a generator and control system (not shown) as described above. The substrate 502 may be used directly by a user or a handle (not shown) may be provided. In addition, the substrate 502 may be a malleable material so that it may deform into the contours of a target site for providing therapeutic contact with the target site or lesion. The substrate 502 may be formed from foam, sponge or other compliant material, or be a compliant container filled with elastomeric or gel. The device may be a sponge or other compliant material that is coated with conductive material and then attached to leads 109. The conductive compliant material becomes the ablation structure. Such an ablation structure would be highly compliant to the target site anatomy. Suitable insulation or mounting structure may be provided to allow the device to be hand held or mounted. Alternatively, the substrate 502 may also be a heat set material. In use, the user may apply pressure directly to the substrate 502 in order to increase contact between the electrode array 101 and the target lesion.

FIG. 60 is a perspective view of a finger-mountable ablation therapy device 550. The ablation device 550 includes an electrode array 101 on a band 555 mounted on a finger 19. Leads 109 attach the electrode array 101 to a generator and control system (not shown) as described above. Insulation is provided between the electrode array 101 and the finger 19. The device 550 may also be sized to fit more than one finger or in different sized fingers such as male and female finger sizes. The band 555 may be elastic or inelastic. Alternatively, the finger mounted device 550 may be configured as part of a glove worn by the user. In use, the user may direct the ablation therapy by pressure or manipulation of the finger 19 under direct visualization and with tactile feedback.

As described above, embodiments of the present invention may be used for ablation of benign, pre-cancerous and early stage lesions that originate within the epithelium and are limited to the mucosal layer of the gastrointestinal tract. Several specific methods will now be described in turn.

In one aspect, there is a method of providing ablation based therapy in a target area having a cervical inlet patch within a portion of the proximal esophagus. The method includes the steps of manipulating a portion of the proximal esophagus to expose the target area and deploying an ablation device into contact with the target area. Next there are the steps of delivering ablative energy to a tissue surface in the target area and then controlling the delivery of ablative energy to the tissue surface and layers of the target area. The manipulating step may also include identifying a cervical inlet patch within the target area.

The method may also include additional steps such as continuing the manipulating step to expose the target area during the delivering and controlling steps; removing debris from the target area after the controlling step; removing debris from the target area after performing the controlling step more than once or evaluating the target area after the delivering energy step.

Additionally, the method may also include a controlling step for delivery of an energy density within the range of 10-15 J/cm² or, sufficient ablative energy to achieve ablation in one fraction of the tissue target surface and delivering insufficient ablative energy to achieve ablation to another fraction of the target tissue surface, delivering ablative energy without an electrode structure penetrating tissue in the target area or controlling the delivery of ablative energy within the target tissue surface to provide sufficient treatment to achieve ablation within the cereal inlet patch and yet provide insufficient energy to other tissue layers beneath the cervical inlet patch.

The method of treating a cervical inlet patch may also include controlling the delivery of ablative energy across the surface and into tissue layers in the target area is such that some fraction of the tissue volume is ablated and another fraction of the tissue volume is not ablated. Alternatively, the method may include controlling the delivery of energy into target tissue layers consists of ablating: a fraction of tissue in the epithelial layer of the cervical inlet patch; a fraction of tissue in the epithelial layer and the lamina propria of the cervical inlet patch; a fraction of cervical inlet patch tissue in the epithelial layer, the lamina propria, and the muscularis mucosae; a fraction of cervical inlet patch tissue in the epithelial layer, the lamina propria, the muscularis mucosae, and the submucosa; and/or delivering energy in an ablation pattern configured to conform to a cervical inlet patch.

In additional aspects, the controlling step includes adjusting the controlling step based on a feedback control of the energy delivery to provide any of a specific power, a power density, an energy level, an energy density, a circuit impedance, target tissue temperature, a number of applications of energy, or a pressure of application against the tissue. The deploying step may also include moving the ablation structure into therapeutic contact with the target area prior to the delivering energy step. In this context, the moving step may also include expanding an expandable member to enhance the therapeutic contact with the target tissue. In addition, the moving step may include operating a deflection mechanism to enhance the therapeutic contact with the target tissue.

In another alternative method, there is a method of providing ablation based therapy to a target area in a stomach containing intestinal metaplasia, intra-epithelial neoplasia, and/or early gastric cancer, hereafter referred to as “abnormal gastric tissue.” The method includes the steps of manipulating a portion of the stomach to expose the target area and then deploying an ablation device into contact with the target area. Next, there are the steps of delivering ablative energy to a tissue surface in the target area controlling the delivery of ablative energy to the tissue surface and layers of the target area. In one aspect, the manipulating step includes identifying the region of abnormal gastric tissue within the target area after the manipulating step.

In another aspect, the method of treating abnormal gastric tissue may include continuing the manipulating step to expose the target area during the delivering and controlling steps; removing debris from the target area after the controlling step; and removing debris from the target area after performing the controlling step more than once and evaluating the target area after the delivering energy step.

In other embodiments, the delivering step includes delivering ablative energy without an electrode structure penetrating tissue in the target area or delivering energy in an ablation pattern configured to conform to the region of abnormal gastric tissue within the target area. In still other embodiments, the advancing step includes moving the ablation structure into therapeutic contact with the target area prior to the delivering energy step. Moving the ablation structure may include, for example, expanding an expandable member to enhance the therapeutic contact with the target tissue or operating a deflection mechanism to enhance the therapeutic contact with the target tissue.

In other aspects of the method of treating abnormal gastric tissue, the controlling step delivers an energy density of more than 10 J/cm² or higher. In other aspects, the controlling step includes delivering sufficient ablative energy to achieve ablation in one fraction of the tissue target surface and delivering insufficient ablative energy to achieve ablation to another fraction of the target tissue surface. In still another variation, the controlling step includes controlling the delivery of ablative energy from the target tissue surface with sufficient energy to achieve ablation within the region of abnormal gastric tissue within the target area and insufficient energy is delivered to other target tissue layers beneath the region of abnormal gastric tissue within the target area. In still another variation, controlling the delivery of ablative energy across the surface and into tissue layers in the target area is such that some fraction of the tissue volume is ablated and another fraction of the tissue volume is not ablated. In another aspect, controlling the delivery of energy into target tissue layers consists of ablating a fraction of tissue in the epithelial layer of the region of abnormal gastric tissue within the target area. In still another aspect, controlling the delivery of energy into target tissue layers consists of ablating a fraction of tissue in the epithelial layer and the lamina propria of the region of abnormal gastric tissue within the target area. In another aspect, controlling the delivery of energy into the tissue layers consists of ablating a fraction of the region of abnormal gastric tissue within the target area tissue in the epithelial layer, the lamina propria, and the muscularis mucosae. In another variation, controlling the delivery of energy into tissue layers consists of ablating a fraction of abnormal gastric tissue within the target area in the epithelial layer, the lamina propria, the muscularis mucosae, and the submucosa. In still another variation, the controlling step includes adjusting the controlling step based on a feedback control of the energy delivery to provide any of a specific power, a power density, an energy level, an energy density, a circuit impedance, target tissue temperature, a number of applications of energy, or a pressure of application against the tissue.

In another alternative method, there is a method of providing ablation based therapy to a target area in an esophagus having a region of a squamous intra-epithelial neoplasia and/or early cancer of the esophagus, hereafter referred to as “abnormal esophageal tissue”. The method of providing ablation based therapy to a target area in an esophagus having a region of abnormal esophageal tissue within the target area includes the step of identifying the region of a abnormal esophageal tissue within the target area. Next, there is the step of advancing an ablation device into contact with the target area and delivering ablative energy to a tissue surface in the target area. Next, there is the step of controlling the delivery of ablative energy to the tissue surface and layers of the target area.

The method of treating abnormal esophageal tissue may also include additional steps such as: removing debris from the target area after the controlling step, removing debris from the target area after performing the controlling step more than once or evaluating the target area after the delivering energy step.

In still other embodiments, the advancing step includes moving the ablation structure into therapeutic contact with the target area prior to the delivering energy step. Moving the ablation structure may include, for example, expanding an expandable member to enhance the therapeutic contact with the target tissue or operating a deflection mechanism to enhance the therapeutic contact with the target tissue.

In one aspect, the delivery step includes delivering energy nearly circumferentially about the esophagus to a region of abnormal esophageal tissue within a nearly circumferential target area in the esophagus. Alternatively, the delivering energy step includes delivering energy less than circumferentially about the esophagus to a region of a squamous intra-epithelial neoplasia within a less than circumferential target area in the esophagus. In another aspect, the delivering ablative energy step includes delivering ablative energy without an electrode structure penetrating tissue in the target area. In still another variation, the delivering energy step includes delivery in an ablation pattern configured to conform to the region of abnormal esophageal tissue within the target area.

In still another variation of the method to treat abnormal esophageal tissue within a target region, the controlling step delivers a power density in the range of 10 to 15 J/cm². In still other variations, the controlling step includes delivering sufficient ablative energy to achieve ablation in one fraction of the tissue target surface and delivering insufficient ablative energy to achieve ablation to another fraction of the target tissue surface. In still another variation, the controlling step is controlling the delivery of ablative energy from the target tissue surface with sufficient energy to achieve ablation within the region of a abnormal esophageal tissue in the target area and insufficient energy is delivered to other target tissue layers beneath the region of abnormal esophageal tissue within the target area. In still another variation, the controlling step is controlling the delivery of ablative energy across the surface and into tissue layers in the target area is such that some fraction of the tissue volume is ablated and another fraction of the tissue volume is not ablated. In still another variation, the controlling step is controlling the delivery of energy into target tissue layers consists of ablating a fraction of tissue in the epithelial layer of the region of abnormal esophageal tissue within the target area. In still another variation, the controlling step is controlling the delivery of energy into target tissue layers consists of ablating a fraction of tissue in the epithelial layer and the lamina propria of the region of abnormal esophageal tissue within the target area. In still another variation, the controlling step is controlling the delivery of energy into the tissue layers consists of ablating a fraction of the region of abnormal esophageal tissue within the target area tissue in the epithelial layer, the lamina propria, and the muscularis mucosae. In still another variation, the controlling step is controlling the delivery of energy into tissue layers consists of ablating a fraction of abnormal esophageal tissue within the target area in the epithelial layer, the lamina propria, the muscularis mucosae, and the submucosa. In still another variation, there is a step of adjusting the controlling step based on a feedback control of the energy delivery to provide any of a specific power, a power density, an energy level, an energy density, a circuit impedance, target tissue temperature, a number of applications of energy, or a pressure of application against the tissue.

In another alternative embodiment, there is a method of providing ablation based therapy in a target area having a region of leukoplakia within the oral and/or pharyngeal cavity herein after referred to as leukoplakia. The method treating leukoplakia includes the steps of manipulating a portion of the oral and pharyngeal cavity to expose the target area and then deploying an ablation device into contact with the target area. Next, there is the step of delivering ablative energy to a tissue surface in the target area followed by the step of controlling the delivery of ablative energy to the tissue surface and layers of the target area.

The manipulating step may also include identifying a region of leukoplakia within the target area. The delivering step may also include delivering ablative energy without an electrode structure penetrating tissue in the target area. The delivering energy step may also include delivering energy in an ablation pattern configured to conform to a region of leukoplakia. Additionally, the method may include additional steps such as: continuing the manipulating step to expose the target area during the delivering and controlling steps; removing debris from the target area after the controlling step; removing debris from the target area after performing the controlling step more than once or evaluating the target area after the delivering energy step.

Additionally, the method of treating leukoplakia may include a controlling step that delivers a power density within the range of 10-15 J/cm². Alternatively, the controlling step may include delivering sufficient ablative energy to achieve ablation in one fraction of the tissue target surface and delivering insufficient ablative energy to achieve ablation to another fraction of the target tissue surface. In another variation, the controlling step includes controlling the delivery of ablative energy from the target tissue surface with sufficient energy to achieve ablation within the region of leukoplakia and insufficient energy is delivered to other target tissue layers beneath the region of leukoplakia. In another variation, the controlling step includes controlling the delivery of ablative energy across the surface and into tissue layers in the target area is such that some fraction of the tissue volume is ablated and another fraction of the tissue volume is not ablated. In another variation, the controlling step includes controlling the delivery of energy into target tissue layers consists of ablating a fraction of tissue in the epithelial layer of the region of leukoplakia. In another variation, the controlling step includes controlling the delivery of energy into target tissue layers consists of ablating a fraction of tissue in the epithelial layer and the lamina propria of the region of leukoplakia. In another variation, the controlling step includes controlling the delivery of energy into the tissue layers consists of ablating a fraction of the region of leukoplakia tissue in the epithelial layer, the lamina propria, and the muscularis mucosae. In another variation, the controlling step includes controlling the delivery of energy into tissue layers consists of ablating a fraction of the region of leukoplakia tissue in the epithelial layer, the lamina propria, the muscularis mucosae, and the submucosa. In another variation, the controlling step includes adjusting the controlling step based on a feedback control of the energy delivery to provide any of a specific power, a power density, an energy level, an energy density, a circuit impedance, target tissue temperature, a number of applications of energy, or a pressure of application against the tissue.

In an alternative method of treating leukoplakia, the deployment includes moving the ablation structure into therapeutic contact with the target area prior to the delivering energy step. In one aspect, the moving step includes expanding an expandable member to enhance the therapeutic contact with the target tissue. In another aspect, the moving step includes operating a deflection mechanism to enhance the therapeutic contact with the target tissue. In still another aspect, the moving step further includes deforming the ablation structure to at least partially conform to the region of leukoplakia. In still another aspect, there is a step of placing the ablation structure on a finger of a user prior to the advancing step and keeping the ablation structure on the finger of the user during the delivering an controlling steps. This may also be a handheld ablation device. Additionally, the deploying step is performed using a hand held ablation device under direct visualization of the user.

In another alternative method, there is a method of providing ablation based therapy to a target area in a colon and/or rectum having a region of one or more flat-type polyps within the target area. The method includes the steps of manipulating a portion of the colon to expose the target area and deploying an ablation device into contact with the target area. Next, there is the step of delivering ablative energy to a tissue surface in the target area; and then controlling the delivery of ablative energy to the tissue surface and layers of the target area.

In one alternative aspect, the manipulating step includes identifying the region of one or more flat-type polyps within the target area after the manipulating step. The method may also include additional steps such as: continuing the manipulating step to expose the target area during the delivering and controlling steps; removing debris from the target area after the controlling step; removing debris from the target area after performing the controlling step more than once; evaluating the target area after the delivering energy step.

In still other aspects, the delivering ablative energy step includes delivering ablative energy without an electrode structure penetrating tissue in the target area. The delivering energy step may also include delivering energy in an ablation pattern configured to conform to the region of one or more flat-type polyps within the target area. In another aspect, the delivering step includes delivering ablative energy to a tissue surface containing residual flat-type polyp tissue in the target area where a partial or complete polypectomy has been performed.

In another aspect of a method of treating a region of one or more flat-type polyps within the target area, the controlling step delivers a power density of 10 J/cm² or greater. In another variation, the controlling step includes delivering sufficient ablative energy to achieve ablation in one fraction of the tissue target surface and delivering insufficient ablative energy to achieve ablation to another fraction of the target tissue surface. In another variation, the controlling step includes controlling the delivery of ablative energy from the target tissue surface with sufficient energy to achieve ablation within the region of one or more flat-type polyps within the target area and insufficient energy is delivered to other target tissue layers beneath the region of one or more flat-type polyps within the target area. In another variation, the controlling step includes controlling the delivery of ablative energy across the surface and into tissue layers in the target area is such that some fraction of the tissue volume is ablated and another fraction of the tissue volume is not ablated. In another variation, the controlling step includes controlling the delivery of energy into target tissue layers consists of ablating a fraction of tissue in the epithelial layer of the region of one or more flat-type polyps within the target area. In another variation, the controlling step includes controlling the delivery of energy into target tissue layers consists of ablating a fraction of tissue in the epithelial layer and the lamina propria of the region of one or more flat-type polyps within the target area. In another variation, the controlling step includes controlling the delivery of energy into the tissue layers consists of ablating a fraction of the region of one or more flat-type polyps within the target area tissue in the epithelial layer, the lamina propria, and the muscularis mucosae. In another variation, the controlling step includes controlling the delivery of energy into tissue layers consists of ablating a fraction of the region of one or more flat-type polyps within the target area in the epithelial layer, the lamina propria, the muscularis mucosae, and the submucosa. In another variation, the controlling step includes adjusting the controlling step based on a feedback control of the energy delivery to provide any of a specific power, a power density, an energy level, an energy density, a circuit impedance, target tissue temperature, a number of applications of energy, or a pressure of application against the tissue.

In still another variation, the method includes an advancing step with moving the ablation structure into therapeutic contact with the target area prior to the delivering energy step. In one alternative, the moving step includes expanding an expandable member to enhance the therapeutic contact with the target tissue. In another aspect, the moving step includes operating a deflection mechanism to enhance the therapeutic contact with the target tissue.

In another aspect, there is a method of providing ablation based therapy in an anal target area having a region of abnormal anal tissue. As used herein, abnormal anal tissue refers to anal intraepithelial neoplasia and/or early anal cancer. The method includes the steps of manipulating a portion of the anal canal to expose the target area and deploying an ablation device into contact with the target area. Next, there are the steps of delivering ablative energy to a tissue surface in the target area and then controlling the delivery of ablative energy to the tissue surface and layers of the target area. The method may include additional steps such as: continuing the manipulating step to expose the target area during the delivering and controlling steps; removing debris from the target area after the controlling step; removing debris from the target area after performing the controlling step more than once; or evaluating the target area after the delivering energy step. In addition, the manipulating step may include identifying a region of abnormal anal tissue within the target area.

In still other variations, the step of delivering ablative energy includes delivering ablative energy without an electrode structure penetrating tissue in the target area. In another variation, the delivering energy step includes delivering energy in an ablation pattern configured to conform to a region of intraepithelial neoplasia.

In still other aspects, the method of treating abnormal anal tissue includes a method where the controlling step delivers a power density within the range of 10-15 J/cm². In another variation, the controlling step includes delivering sufficient ablative energy to achieve ablation in one fraction of the tissue target surface and delivering insufficient ablative energy to achieve ablation to another fraction of the target tissue surface. In another variation, the controlling step includes controlling the delivery of ablative energy from the target tissue surface with sufficient energy to achieve ablation within the region of abnormal anal tissue and insufficient energy is delivered to other target tissue layers beneath the region of abnormal anal tissue. In another variation, the controlling step includes controlling the delivery of ablative energy across the surface and into tissue layers in the target area is such that some fraction of the tissue volume is ablated and another fraction of the tissue volume is not ablated. In another variation, the controlling step includes controlling the delivery of energy into target tissue layers consists of ablating a fraction of tissue in the epithelial layer of the region of abnormal anal tissue. In another variation, the controlling step includes controlling the delivery of energy into target tissue layers consists of ablating a fraction of tissue in the epithelial layer and the lamina propria of the region of abnormal anal tissue. In another variation, the controlling step includes controlling the delivery of energy into the tissue layers consists of ablating a fraction of the region of abnormal anal tissue in the epithelial layer, the lamina propria, and the muscularis mucosae. In another variation, the controlling step includes controlling the delivery of energy into tissue layers consists of ablating a fraction of the region of abnormal anal tissue in the epithelial layer, the lamina propria, the muscularis mucosae, and the submucosa. In another variation, the controlling step includes adjusting the controlling step based on a feedback control of the energy delivery to provide any of a of a specific power, a power density, an energy level, an energy density, a circuit impedance, target tissue temperature, a number of applications of energy, or a pressure of application against the tissue.

In another variation of the method of treating abnormal anal tissue, the deployment step includes moving the ablation structure into therapeutic contact with the target area prior to the delivering energy step. In another variation, moving step includes expanding an expandable member to enhance the therapeutic contact with the target tissue. In still another embodiment, the moving step includes operating a deflection mechanism to enhance the therapeutic contact with the target tissue. In still another alternative, the moving step includes deforming the ablation structure to at least partially conform to the region of abnormal anal tissue. In another alternative, the method includes placing the ablation structure on a finger of a user prior to the advancing step and keeping the ablation structure on the finger of the user during the delivering an controlling steps. In another alternative, the deploying step is performed using a hand held ablation device under direct visualization.

While most embodiments described herein have made use of radiofrequency energy as an exemplary ablational energy, and consequently have made use of electrodes as an energy transmitting element, it should be understood that these examples are not limiting with regard to energy source and energy delivery or transmitting elements. As also described herein, other forms of energy, as well as cryoablating approaches, may provide for ablation of target areas in such a manner that ablation is fractional or partial, as described herein, where some portions of target area tissue are ablated, and some portions of target area tissue are not substantially ablated.

### Terms and Conventions

Unless defined otherwise, all technical terms used herein have the same meanings as commonly understood by one of ordinary skill in the art of ablational technologies. Specific methods, devices, and materials are described in this application, but any methods and materials similar or equivalent to those described herein can be used in the practice of the present invention. While embodiments of the invention have been described in some detail and by way of exemplary illustrations, such illustration is for purposes of clarity of understanding only, and is not intended to be limiting. Still further, it should be understood that the invention is not limited to the embodiments that have been set forth for purposes of exemplification, but is to be defined only by a fair reading of claims that are appended to the patent application, including the full range of equivalency to which each element thereof is entitled.

1-104. (canceled)

105. A method of providing ablation based therapy in an anal target area having a region of abnormal anal tissue, comprising:
manipulating a portion of the anal canal to expose the target area;
deploying an ablation device into contact with the target area;
delivering ablative energy to a tissue surface in the target area; and
controlling the delivery of ablative energy to the tissue surface and layers of the target area.

106. The method of claim 105, the manipulating step further comprising:
identifying a region of abnormal anal tissue within the target area.

107. The method of claim 105, further comprising:
continuing the manipulating step to expose the target area during the delivering and controlling steps.

108. The method of claim 105, further comprising:
removing debris from the target area after the controlling step.

109. The method of claim 105, further comprising:
removing debris from the target area after performing the controlling step more than once.

110. The method of claim 105 wherein the controlling step delivers a power density within the range of 10-15 J/cm2.

111. The method of claim 105 wherein the delivering ablative energy step comprises delivering ablative energy without an electrode structure penetrating tissue in the target area.

112. The method of claim 105, the controlling step further comprising:
delivering sufficient ablative energy to achieve ablation in one fraction of the tissue target surface and delivering insufficient ablative energy to achieve ablation to another fraction of the target tissue surface.

113. The method of claim 105, the controlling step further comprising:
controlling the delivery of ablative energy from the target tissue surface with sufficient energy to achieve ablation within the region of abnormal anal tissue and insufficient energy is delivered to other target tissue layers beneath the region of abnormal anal tissue.

114. The method of claim 105 wherein controlling the delivery of ablative energy across the surface and into tissue layers in the target area is such that some fraction of the tissue volume is ablated and another fraction of the tissue volume is not ablated.

115. The method of claim 106 wherein controlling the delivery of energy into target tissue layers consists of ablating a fraction of tissue in the epithelial layer of the region of abnormal anal tissue.

116. The method of claim 106 wherein controlling the delivery of energy into target tissue layers consists of ablating a fraction of tissue in the epithelial layer and the lamina propria of the region of abnormal anal tissue.

117. The method of claim 106 wherein controlling the delivery of energy into the tissue layers consists of ablating a fraction of the region of abnormal anal tissue in the epithelial layer, the lamina propria, and the muscularis mucosae.

118. The method of claim 106 wherein controlling the delivery of energy into tissue layers consists of ablating a fraction of the region of abnormal anal tissue in the epithelial layer, the lamina propria, the muscularis mucosae, and the submucosa.

119. The method of claim 105, the delivering energy step further comprising: delivering energy in an ablation pattern configured to conform to a region of intraepithelial neoplasia.

120. The method of claim 105, further comprising: evaluating the target area after the delivering energy step.

121. The method of claim 105, the controlling step further comprising:
adjusting the controlling step based on a feedback control of the energy delivery to provide any of a of a specific, power, a power density, an energy level, an energy density, a circuit impedance, target tissue temperature, a number of applications of energy, or a pressure of application against the tissue.

122. The method of claim 105, the deployment step further comprising:
moving the ablation structure into therapeutic contact with the target area prior to the delivering energy step.

123. The method of claim 122, the moving step further comprising:
expanding an expandable member to enhance the therapeutic contact with the target tissue.

124. The method of claim 122, the moving step further comprising:
operating a deflection mechanism to enhance the therapeutic contact with the target tissue.

125. The method of claim 122 the moving step further comprising:
deforming the ablation structure to at least partially conform to the region of abnormal anal tissue.

126. The method of claim 105 further comprising:
placing the ablation structure on a finger of a user prior to the advancing step and keeping the ablation structure on the finger of the user during the delivering an controlling steps.

127. The method of claim 105 wherein the deploying step is performed using a hand held ablation device under direct visualization.
